(19) United States
(12) Patent Application Publication
(10) Pub. No.: US 2026/0199282 A1
(43) Pub. Date: Jul. 16, 2026

(54) METHODS AND COMPOSITIONS FOR IMPROVING DRIVING PERFORMANCE

(71) Applicant: AXSOME THERAPEUTICS, INC., New York, NY (US)

(72) Inventors: Grace WANG, New York, NY (US); Dan CHEN, New York, NY (US); Lawrence Patrick CARTER, Palo Alto, CA (US)

(21) Appl. No.: 19/134,503

(22) PCT Filed: Nov. 30, 2023
(86) PCT No.: PCT/US2023/081798

§ 371 Date: May 30, 2025

Related U.S. Application Data
(60) Provisional application No. 63/385,539, filed on Nov. 30, 2022.

(51) Int. Cl. A61K 31/27; A61P 25/00; A61P 25/26

(52) U.S. Cl. CPC A61K 31/27; A61P 25/00; A61P 25/26

(57) ABSTRACT

Provided herein according to some embodiments is a method of improving on-the-road driving performance in a subject in need thereof, said method comprising administering to the subject a pharmaceutically effective amount of [R]-2-amino-3-phenylpropylcarbamate (APC) or a pharmaceutically acceptable salt thereof, for example, in a dose of between 37.5 mg and 300 mg of APC or a pharmaceutically acceptable salt thereof, thereby improving on-the-road driving performance in the subject.

### FIELD OF THE INVENTION

The present invention relates to [R]-2-amino-3-phenylpropylcarbamate (APC) or a pharmaceutically acceptable salt thereof, and methods of using to improve on-the-road driving performance.

### BACKGROUND

Excessive sleepiness, also known as excessive daytime sleepiness (EDS), is a symptom that is defined as difficulty in maintaining wakefulness and an increased propensity to fall asleep throughout the day, even in inappropriate circumstances and in situations that interfere with activities of daily living or pose safety risks. Worldwide, the prevalence of EDS ranges from 10% to 33% (Hayley 2014; Jaussent 2017; Joo 2009; Wu 2012).

By virtue of the severity, chronicity, pervasiveness, and lack of response to usual countermeasures, EDS profoundly affects those afflicted, and no amount of extra sleep will lessen its debilitating effect. Regardless of the underlying pathophysiology of EDS, the symptom manifestations and clinical consequences of EDS are similar. The most disabling consequences include undesired sleep episodes, reduced attention, cognitive impairment, compromised performance, effects on mood, and an increased risk for motor vehicle accidents and accidents in the home and/or workplace.

Narcolepsy is a chronic neurological disorder characterized by excessive daytime sleepiness (EDS) (Kornum et al., 2017; Szabo, Thorpy, Mayer, Peever, & Kilduff, 2019). Patients with narcolepsy often experience negative effects on daily functioning (Flores, Villa, Black, Chervin, & Witt, 2016), including impaired driving performance (Findley et al., 1995; Kotterba et al., 2004). Patients with narcolepsy are also at higher risk for motor vehicle accidents (MVAs) and resulting hospitalizations (Liu, Perez, & Lau, 2018; Philip et al., 2010; Pizza et al., 2015; Tzeng et al., 2019). For example, in a case-control study of MVAs occurring during the preceding year, the odds of having any MVA were ~3 times greater (and the odds of sleepiness-related MVA>8 times greater) in drivers with narcolepsy or hypersomnia compared with controls (Philip et al., 2010). Experimental evidence suggests that treatment with modafinil improves some measures of on-road (Philip et al., 2014) and simulated driving (Kotterba et al., 2004; Sagaspe et al., 2019) performance in patients with narcolepsy or hypersomnia. In addition, two epidemiologic studies showed that long-term treatment with modafinil or psychostimulants reduced the risk for MVAs (Pizza et al., 2015; Tzeng et al., 2019). While reduced sleep latency, as measured with the Maintenance of Wakefulness Test, has been shown to be significantly correlated with sleepiness-related MVAs and near misses in a population of patients with diverse sleep disorders (Philip et al., 2021), a reliable predictor of fitness to drive in patients with narcolepsy specifically is still lacking.

Similarly, the excessive sleepiness, inattention, and fatigue associated with OSA may significantly increase the risk of driving accidents, among other impairments. Motor vehicle crashes are 2 to 3 times more common among patients with OSA than without OSA; this represents an impact on morbidity and mortality that is similar to the cardiovascular sequelae of OSA (George 2007). A marked reduction was observed in the incidence of real crashes, near-misses, and crash-related events in a driving simulator after the initiation of continuous positive airway pressure treatment, indicating that successful OSA treatment improves driving simulator performance and decreases motor vehicle crashes (Tregear 2010). Improvement in driving performance has also been demonstrated in studies that assessed the effect of simulants in the setting of simulated driving and on-road driving (Ramaekers 2012). In newly diagnosed, treatment-naive OSA patients who had excessive sleepiness, 2 weeks of treatment with armodafinil improved several performance measures in a simulated driving test (Kay and Feldman 2013).

(R)-2-amino-3-phenylpropyl carbamate (APC) is a phenylalanine analog that has been demonstrated to be useful in the treatment of a variety of disorders, including excessive daytime sleepiness, cataplexy, narcolepsy, fatigue, depression, bipolar disorder, fibromyalgia, and others. See, for example, U.S. Pat. Nos. 8,232,315; 8,440,715; 8,552,060; 8,623,913; 8,729,120; 8,741,950; 8,895,609; 8,927,602; 9,226,910; and 9,359,290; and U.S. Publication Nos. 2012/0004300 and 2015/0018414. Methods for producing APC (which also has other names) and related compounds can be found in U.S. Pat. Nos. 5,955,499; 5,705,640; 6,140,532 and 5,756,817. All of the above patents and applications are hereby incorporated by reference in their entireties for all purposes.

The present invention overcomes shortcomings in the art by providing methods of improving on-the-road driving performance in a subject in need thereof.

### SUMMARY OF THE INVENTION

The present invention relates to the development of methods of improving on-the-road driving performance in a subject in need thereof.

Accordingly, one aspect of the invention relates to a method of improving on-the-road driving performance in a subject in need thereof, said method comprising administering to the subject a pharmaceutically effective amount of [R]-2-amino-3-phenylpropylcarbamate (APC) or a pharmaceutically acceptable salt thereof, thereby improving on-the-road driving performance in the subject.

Another aspect of the invention relates to a method of improving on-the-road driving performance in a subject in need thereof, said method comprising administering to the subject a pharmaceutically effective amount of [R]-2-amino-3-phenylpropylcarbamate (APC) or a pharmaceutically acceptable salt thereof. In some embodiments, the daily dose of solriamfetol is 75 mg or 150 mg.

In some embodiments, the subject has excessive daytime sleepiness. In some embodiments, the excessive daytime sleepiness is associated with narcolepsy or obstructive sleep apnea.

In some embodiments, the on-the-road driving performance is assessed by measuring the standard deviation of lateral position (SDLP), standard deviation of speed, and/or number of lane drifts while operating a vehicle. In some embodiments, the on-the-road driving performance is measured over the course of about 30 minutes to about 120 minutes. In some embodiments, the on-the-road driving performance is assessed from about 1 hour after providing APC to the subject, to about 12 hours after administering APC to the subject. In some embodiments, the on-the-road driving performance is assessed by measuring the SDLP improves from about 1.0 cm to about 15 cm after providing APC to the subject.

In some embodiments, the subject is a human.

These and other aspects of the invention are set forth in more detail in the description of the invention below.

### BRIEF DESCRIPTION OF THE DRAWINGS

FIG. 1A. Study design for investigating effects of solriamfetol on on-the-road driving in participants with narcolepsy; FU, follow-up; THAT, Toronto Hospital Alertness Test; V, visit.

FIG. 1B. Overview of study design for investigating effects of solriamfetol on on-the-road driving in participants with narcolepsy; FU=follow-up; PBO=placebo; PVT=Psychomotor Vigilance Test; V=visit.

FIGS. 2A-2D. Individual driving performance and symmetry analysis in narcolepsy study. (A) Standard Deviation of Lateral Position (SDLP) difference from placebo by participant at 2 hours (n=22) and 6 hours (n=21) post-dose. (B) Symmetry analysis of SDLP difference scores at 2 hours post-dose: percentage (out of n=22) with improvement vs. impairment of driving performance with solriamfetol compared with placebo, at thresholds increasing from 1.0 cm to 3.5 cm. (C) SDLP at 2 hours post-dose by participant (n=22). (D) SDLP at 6 hours post-dose by participant (n=21). SDLP, standard deviation of lateral position.

FIG. 3. SDLP post-dose analysis at 2 hours and 6 hours timepoint of modified Intent-to-Treat (mITT) Population from narcolepsy study.

FIG. 4. Overview of study design for investigating effects of solriamfetol on on-the-road driving in participants with obstructive sleep apnea (OSA); FU=follow-up; PBO=placebo; PVT=Psychomotor Vigilance Test; V=visit.

FIG. 5. SDLP post-dose analysis at 2 hours and 6 hours timepoint of modified Intent-to-Treat (mITT) Population from OSA study. Data presented includes SDLP change from placebo for all subjects including those that did not complete the driving test.

FIGS. 6A-6B. Predicted and actual task effectiveness across PVT test sessions for participants with OSA (A) Placebo; (B) Solriamfetol (APC), ^{a}Actual task effectiveness between drug conditions was compared with modeled healthy control task effectiveness using paired-samples t test, controlling for drug condition and test session. P values are nominal. OSA, obstructive sleep apnea; PVT, psychomotor vigilance task; SD, standard deviation.

FIGS. 7A-7B. Predicted and actual task effectiveness across PVT test sessions for participants with OSA (A) Placebo; (B) Solriamfetol (APC), ^{a}Actual task effectiveness between drug conditions was compared with modeled healthy control task effectiveness using paired-samples t test, controlling for drug condition and test session. P values are nominal. OSA, obstructive sleep apnea; PVT, psychomotor vigilance task; SD, standard deviation.

FIGS. 8A-8C. Sleep outcomes in participants with OSA. (A) OSA time in Bed; (B) OSA Total Time in Bed (C) OSA Daily Sleep Intervals.

FIGS. 9A-9C. Sleep outcomes in participants with Narcolepsy. (A) Narcolepsy time in Bed; (B) Narcolepsy Total Time in Bed (C) Narcolepsy Daily Sleep Intervals.

### DETAILED DESCRIPTION

The present invention will now be described in more detail with reference to the accompanying drawings, in which preferred embodiments of the invention are shown. This invention may, however, be embodied in different forms and should not be construed as limited to the embodiments set forth herein. Rather, these embodiments are provided so that this disclosure will be thorough and complete, and will fully convey the scope of the invention to those skilled in the art. In addition, any references cited herein are incorporated by reference in their entireties.

Unless otherwise defined, all technical and scientific terms used herein have the same meaning as commonly understood by one of skill in the art to which this invention belongs. The terminology used in the description of the invention herein is for the purpose of describing particular embodiments only and is not intended to be limiting of the invention. All publications, patent applications, patents, patent publications and other references cited herein are incorporated by reference in their entireties for the teachings relevant to the sentence and/or paragraph in which the reference is presented.

Unless the context indicates otherwise, it is specifically intended that the various features of the invention described herein can be used in any combination.

Moreover, the present invention also contemplates that in some embodiments of the invention, any feature or combination of features set forth herein can be excluded or omitted.

To illustrate, if the specification states that a complex comprises components A, B and C, it is specifically intended that any of A, B or C, or a combination thereof, can be omitted and disclaimed singularly or in any combination.

As used in the description of the invention and the appended claims, the singular forms “a,” “an,” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount of polypeptide, dose, time, temperature, enzymatic activity or other biological activity and the like, is meant to encompass variations of ±10%, 5%, 1%, 0.5%, or even ±0.1% of the specified amount.

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

The term “therapeutically effective amount” or “effective amount,” as used herein, refers to that amount of a composition, compound, or agent of this invention that imparts a modulating effect, which, for example, can be a beneficial effect, to a subject afflicted with a disorder, disease or illness, including improvement in the condition of the subject (e.g., in one or more symptoms), delay or reduction in the progression of the condition, prevention or delay of the onset of the disorder, and/or change in clinical parameters, disease or illness, etc., as would be well known in the art. For example, a therapeutically effective amount or effective amount can refer to the amount of a composition, compound, or agent that improves a condition in a subject by at least 5%, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 100%.

“Pharmaceutically acceptable carrier” (sometimes referred to as a “carrier”) refers to a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

The term “modulate,” “modulates,” or “modulation” refers to enhancement (e.g., an increase) or inhibition (e.g., a decrease) in the specified level or activity.

The term “enhance” or “increase” refers to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified level and/or activity of at least about 1%, 5%, 10%, 15%, 25%, 35%, 40%, 50%, 60%, 75%, 80%, 90%, 95% or more.

“Inhibit” or “reduce” or grammatical variations thereof as used herein refers to a decrease or diminishment in the specified level or activity of at least about 1, 5, 10, 15%, 25%, 35%, 40%, 50%, 60%, 75%, 80%, 90%, 95% or more. In particular embodiments, the inhibition or reduction results in little or essentially no detectible activity (at most, an insignificant amount, e.g., less than about 10% or even 5%).

“Treat,” “treating” and similar terms as used herein in the context of treating a subject refer to providing medical and/or surgical management of a subject. Treatment may include, but is not limited to, administering an agent or composition (e.g., a pharmaceutical composition) to a subject. Treatment is typically undertaken in an effort to alter the course of a disease (which term is used to indicate any disease, disorder, syndrome, or undesirable condition warranting or potentially warranting therapy) in a manner beneficial to the subject. The effect of treatment may include reversing, alleviating, reducing severity of, delaying the onset of, curing, inhibiting the progression of, and/or reducing the likelihood of occurrence or recurrence of the disease or one or more symptoms or manifestations of the disease. A therapeutic agent may be administered to a subject who has a disease or is at increased risk of developing a disease relative to a member of the general population. In some embodiments a therapeutic agent may be administered to a subject who has had a disease but no longer shows evidence of the disease. The agent may be administered e.g., to reduce the likelihood of recurrence of evident disease. A therapeutic agent may be administered prophylactically, i.e., before development of any symptom or manifestation of a disease. “Prophylactic treatment” refers to providing medical and/or surgical management to a subject who has not developed a disease or does not show evidence of a disease in order, e.g., to reduce the likelihood that the disease will occur, delay the onset of the disease, or to reduce the severity of the disease should it occur. The subject may have been identified as being at risk of developing the disease (e.g., at increased risk relative to the general population or as having a risk factor that increases the likelihood of developing the disease.

Grammatical variations of “administer,” “administration,” and “administering” to a subject include any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, parenteral (e.g., subcutaneous, intravenous, intramuscular, intra-articular, intra-synovial, intrasternal, intrathecal, intraperitoneal, intrahepatic, intralesional, and intracranial injections or infusion techniques), and the like. “Concurrent administration,” “administration in combination,” “simultaneous administration,” or “administered simultaneously” as used herein, means that the compounds are administered at the same point in time, overlapping in time, or one following the other. In the latter case, the two compounds are administered at times sufficiently close that the results observed are indistinguishable from those achieved when the compounds are administered at the same point in time. “Systemic administration” refers to the introducing or delivering to a subject an agent via a route which introduces or delivers the agent to extensive areas of the subject's body (e.g., greater than 50% of the body), for example through entrance into the circulatory or lymph systems. By contrast, “local administration” refers to the introducing or delivery to a subject an agent via a route which introduces or delivers the agent to the area or area immediately adjacent to the point of administration and does not introduce the agent systemically in a therapeutically significant amount. For example, locally administered agents are easily detectable in the local vicinity of the point of administration but are undetectable or detectable at negligible amounts in distal parts of the subject's body. Administration includes self-administration and the administration by another.

“Pharmaceutically acceptable,” as used herein, means a material that is not biologically or otherwise undesirable, i.e., the material can be administered to an individual along with the compositions of this invention, without causing substantial deleterious biological effects or interacting in a deleterious manner with any of the other components of the composition in which it is contained. The material would naturally be selected to minimize any degradation of the active ingredient and to minimize any adverse side effects in the subject, as would be well known to one of skill in the art (see, e.g., Remington's Pharmaceutical Science; 21st ed. 2005).

“Concurrently” means sufficiently close in time to produce a combined effect (that is, concurrently can be simultaneously, or it can be two or more events occurring within a short time period before or after each other). In some embodiments, the administration of two or more compounds “concurrently” means that the two compounds are administered closely enough in time that the presence of one alters the biological effects of the other. The two compounds can be administered in the same or different formulations or sequentially. Concurrent administration can be carried out by mixing the compounds prior to administration, or by administering the compounds in two different formulations, for example, at the same point in time but at different anatomic sites or using different routes of administration.

The present invention is based, in part, on methods of using (R)-2-amino-3-phenylpropyl carbamate (APC)(also known as solriamfetol, and previously known as JZP-110, ADX-N05, R228060, and YKP10A)) for improving on-the-road driving performance in a subject in need thereof.

Accordingly, one aspect of the invention relates to a method of improving on-the-road driving performance in a subject in need thereof, said method comprising administering to the subject a pharmaceutically effective amount of APC or a pharmaceutically acceptable salt thereof, thereby improving on-the-road driving performance in the subject. In some embodiments, the method comprises administering APC to the subject at a daily dose of about 37.5 mg to about 300 mg. As described herein, methods identify the safety and tolerability of solriamfetol in on-the road driving performance.

A “disorder amenable to treatment with solriamfetol” or a “disorder treatable with solriamfetol” refers to any disorder in which administration of solriamfetol to a subject results in the treatment of one or more symptoms of the disorder in the subject. Example disorders amenable to treatment with solriamfetol include narcolepsy, cataplexy, excessive daytime sleepiness, obstructive sleep apnea, drug addiction, sexual dysfunction, fatigue, fibromyalgia, attention deficit/hyperactivity disorder (ADHD), cognitive impairment and/or cognitive dysfunction, restless legs syndrome, depression, bipolar disorder, obesity, or binge eating disorder. In some embodiments, the disorders amenable to treatment with solriamfetol include narcolepsy, excessive daytime sleepiness, obstructive sleep apnea, cognitive impairment, attention deficit/hyperactivity disorder, or binge eating disorder. See, for example, U.S. Pat. Nos. 8,232,315; 8,440,715; 8,552,060; 8,623,913; 8,729,120; 8,741,950; 8,895,609; 8,927,602; 9,226,910; and 9,359,290; and U.S. Publication Nos. 2012/0004300 and 2015/0018414. All of the above patents and applications are hereby incorporated by reference in their entireties for all purposes.

“Excessive daytime sleepiness” or “EDS” refers to persistent sleepiness at a time when the individual would be expected to be awake and alert, even during the day after apparently adequate or even prolonged nighttime sleep. EDS may be the result of a sleep disorder or a symptom of another underlying disorder such as narcolepsy, sleep apnea, circadian rhythm sleep disorder, or idiopathic hypersomnia. While the name includes “daytime,” it is understood that the sleepiness may occur at other times that the subject should be awake, such as nighttime or other times, e.g., if the subject is working nightshift. It is also understood that EDS is medically distinct from fatigue and disorders associated with fatigue.

The methods of the invention may be effective no matter the cause of the EDS, but in some embodiments of the invention, the EDS is associated with narcolepsy or obstructive sleep apnea (OSA). In some embodiments, the EDS is associated with depression. In other embodiments, the cause of the EDS may be, without limitation, central nervous system (CNS) pathologic abnormalities, stroke, idiopathic CNS hypersomnia; sleep deficiency, other sleep apnea, insufficient nocturnal sleep, chronic pain, acute pain, Parkinson's disease, urinary incontinence, multiple sclerosis fatigue, attention deficit hyperactivity disorder (ADHD), Alzheimer's disorder, bipolar disorder, cardiac ischemia; misalignments of the body's circadian pacemaker with the environment, jet lag, shift work, or sedating drugs.

The methods of the invention may also be used to increase wakefulness and/or alertness in a subject in need thereof in on-the road driving.

The structure of the free base of APC is given below as Formula I.

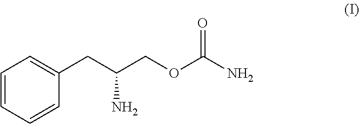 [SMILES: NC(=O)OC[C@H](N)Cc1ccccc1]

[R]-2-amino-3-phenylpropylcarbamate hydrochloride (APC-HCl, also referred to herein as solriamfetol) and related compounds can be found in U.S. Pat. Nos. 10,829,443, 5,955,499; 5,705,640; 6,140,532 and 5,756,817. All of the above patents and applications are hereby incorporated by reference in their entireties for all purposes.

In one embodiment, the methods detailed herein provide a subject to whom APC or a pharmaceutically acceptable salt thereof is administered with improved on-the-road driving performance. The on-the-road driving performance can be assessed by measuring the standard deviation of lateral position (SDLP), standard deviation of speed, and/or number of lane drifts while operating a vehicle. In an example embodiment, lane drifts are defined as greater than 50 cm, 60 cm, 70 cm, 80 cm, 90 com, 100 cm or more from the absolute lateral position within a time window. In some embodiments, the time window can be 5, 6, 7, 8, 9, 10 or more seconds. Monitoring can be performed over the time period in which APC is administered to the subject, which may be over days, weeks, or months, with monitoring over any interval in that time frame, including hourly, daily, weekly, monthly or any time range therein.

In some embodiments, the on-the-road driving performance is measured over the course of about 30 minutes to about 120 minutes, for example, 30, 40, 50, 60, 70, 80, 90, 100, 110, or about 120 minutes. In some embodiments, on-the-road driving performance is assessed from about 1 hour after providing APC or a pharmaceutically acceptable salt thereof to the subject, to about 12 hours after administering APC or a pharmaceutically acceptable salt thereof to the subject, for example 60 minutes, 70 minutes, 80 minutes, 90 minutes, 100 minutes, 110 minutes, 120 minutes, 130 minutes, 140 minutes, 150 minutes, 160 minutes, 170 minutes, 3 hours, 4 hours, 5 hours, 6 hours, 7 hours, 8 hours, 9 hours, 10 hours, 11 hours or about 12 hours after administering APC or a pharmaceutically acceptable salt thereof to the subject. The on-the-road driving performance can be assessed at more than one time, for example, at two hours (e.g., between 1 to 3 hours) and at 6 hours (between 5 to 7 hours) after providing APC or a pharmaceutically acceptable salt thereof to the subject. In some embodiments, the on-the-road driving performance is assessed after a steady state dose of APC or a pharmaceutically acceptable salt thereof is achieved.

Additional assessments for the effects of APC or a pharmaceutically acceptable salt thereof administered to the subject can include measures of attention, reaction time (RT), sustained attention and vigilance. Psychomotor performance can also be assessed, for example using a psychomotor vigilance test (PVT). In some embodiments, errors of commission on the PVT can be used as a measure of impulsivity.

Monitoring can be performed over the time period in which APC is administered to the subject, which may be over days, weeks, or months, with monitoring over any interval in that time frame, including hourly, daily, weekly, monthly or any time range therein.

A daily dose of about 1 to about 2000 mg of APC or a pharmaceutically acceptable salt thereof may be administered to accomplish the therapeutic results disclosed herein. For example, a daily dosage of about 1-1000 mg, e.g., about 20-500 mg, in single or divided doses, is administered. In some embodiments, the daily dose may be about 0.01 to about 150 mg/kg body weight, e.g., about 0.2 to about 18 mg/kg body weight. In some embodiments, the dose contains about 1 mg to about 1000 mg of the drug or any range or value therein, e.g., about 10 mg to about 500 mg, e.g., about 37.5 mg, about 75 mg, about 150 mg, or about 300 mg. For example, in certain such embodiments, the total amount of drug may be selected from about 10, 20, 30, 37.5, 40, 50, 60, 70, 75, 80, 90, 100, 125, 150, 175, 200, 225, 250, 275, 300, or any range therein.

When the compound is in a salt form, the amount of compound may be equivalent to the amount of free base compound, i.e., not in salt form. A dose is “equivalent to” a 37.5 mg, 75 mg, or 150 mg of APC, if the weight of the APC base (the “active moiety”) in the formulation is 37.5 mg, 75 mg, or 150 mg, respectively, regardless of the weight of the APC salt. Thus, the weight of the APC salt may be greater than 37.5 mg, 75 mg, or 150 mg, respectively, in the formulation. Where APC is provided in the form of APC-HCl salt (i.e., solriamfetol), a dose of 37.5 mg APC is equivalent to 44.7 mg (or 44.65 mg) of APC-HCl; a dose of 75 mg APC is equivalent to 89.3 mg of APC-HCl; and a dose of 150 mg APC is equivalent to 178.5 mg of APC-HCl.

In one embodiment of the invention, APC or a pharmaceutically acceptable salt thereof is administered to the subject as needed to treat a disorder. The compound can be administered continuously or intermittently. In one embodiment, the compound is administered to the subject more than once a day, e.g., 2, 3, or 4 times per day, or once every 1, 2, 3, 4, 5, 6, or 7 days. In another embodiment, the compound is administered to the subject no more than once a week, e.g., no more than once every two weeks, once a month, once every two months, once every three months, once every four months, once every five months, once every six months, or longer. In a further embodiment, the compound is administered using two or more different schedules, e.g., more frequently initially (for example to build up to a certain level, e.g., once a day or more) and then less frequently (e.g., once a week or less). In other embodiments, the compound can be administered by any discontinuous administration regimen. In one example, the compound can be administered not more than once every three days, every four days, every five days, every six days, every seven days, every eight days, every nine days, or every ten days, or longer. The administration can continue for one, two, three, or four weeks or one, two, or three months, or longer. Optionally, after a period of rest, the compound can be administered under the same or a different schedule. The period of rest can be one, two, three, or four weeks, or longer, according to the pharmacodynamic effects of the compound on the subject. In another embodiment the compound can be administered to build up to a certain level, then maintained at a constant level and then a tailing dosage.

In one aspect of the invention, APC or a pharmaceutically acceptable salt thereof is delivered to a subject concurrently with an additional therapeutic agent. The additional therapeutic agent can be delivered in the same composition as the compound or in a separate composition. The additional therapeutic agent can be delivered to the subject on a different schedule or by a different route as compared to the compound. The additional therapeutic agent can be any agent that provides a benefit to the subject. Further agents include, without limitation, stimulants, anti-psychotics, anti-depressants, agents for neurological disorders, and chemotherapeutic agents. One therapeutic agent that can be administered during the same period is Xyrem®, sold commercially by Jazz Pharmaceuticals, which is used to treat narcolepsy and cataplexy. See U.S. Pat. Nos. 8,952,062 and 9,050,302.

The present invention finds use in research as well as veterinary and medical applications. Suitable subjects are generally mammalian subjects. The term “mammal” as used herein includes, but is not limited to, humans, non-human primates, cattle, sheep, goats, pigs, horses, cats, dog, rabbits, rodents (e.g., rats or mice), etc. Human subjects include neonates, infants, juveniles, adults, and geriatric subjects.

The subject can be a subject “in need of” the methods of the present invention, e.g., in need of the therapeutic effects of the inventive methods. For example, the subject can be a subject that is experiencing a disorder amenable to treatment with APC or a pharmaceutically acceptable salt thereof, is suspected of having a disorder amenable to treatment with APC or a pharmaceutically acceptable salt thereof, and/or is anticipated to experience a disorder amenable to treatment with APC or a pharmaceutically acceptable salt thereof, and the methods and compositions of the invention are used for therapeutic and/or prophylactic treatment.

### APC Salts

The methods of the present invention may be carried out using compounds, formulations and unit dosage forms provided herein. In some embodiments, the formulations and dosage forms may include pharmaceutically acceptable salts of APC (“APC salt”), which also includes hydrates, solvates, clathrates, inclusion compounds, and complexes thereof.

In some embodiments of the invention, the APC salt is a hydrochloride salt APC-HCl). However, suitable salts of APC also include, without limitation, acetate, adipate, alginate, aspartate, benzoate, butyrate, citrate, fumarate, glycolate, hemisulfate, heptanoate, hexanoate, hydrobromide, hydroiodide, 2-hydroxyethanesulfonate, lactate, maleate, malonate, methanesulfonate, nicotinate, nitrate, oxalate, palmoate, pectinate, persulfate, hydroxynapthoate, pivalate, propionate, salicylate, succinate, sulfate, tartrate, thiocyanate, tosylate and undecanoate. Other acids, such as oxalic, while not in themselves pharmaceutically acceptable, can be employed in the preparation of salts useful as intermediates in obtaining the compound of the invention and their pharmaceutically acceptable acid addition salts. APC salts include those having quaternization of any basic nitrogen-containing group therein.

The discussion herein is, for simplicity, provided without reference to the addition of deuterium atoms, but the APC salts may further include non-ordinary isotopes. Those skilled in the art will appreciate that the APC salt can contain one or more asymmetric centers and thus occur as racemates and racemic mixtures and single optical isomers. In embodiments of the present invention, the APC salt stereoisomer is preferred, but formulations according to embodiments of the invention may include both (R) and (S) isomers in a racemic mixture, or in any ratio of the isomers. In particular embodiments, the (R)-2-amino-3-phenylpropyl carbamate salt stereoisomer is present at a greater concentration than the (S)-2-amino-3-phenylpropyl carbamate salt stereoisomer, and in some embodiments, the formulation includes the 2-amino-3-phenylpropyl carbamate salt as a substantially enantiomerically pure (R)-2-amino-3-phenylpropyl carbamate salt stereoisomer such as having an enantiomeric excess of greater than 80%, 90%, 95%, or 99%. In some embodiments, the (R)-2-amino-3-phenylpropyl carbamate salt is enantiomerically pure, and in some cases is enantiomerically pure (R)-2-amino-3-phenylpropyl carbamate hydrochloride. When the (R)-2-amino-3-phenylpropyl carbamate salt is referenced specifically, it is understood that the dosage (e.g., 37.5 mg or 75 mg) refers to the equivalent weight of the (R) enantiomer only.

The APC salt(s) may be obtained or synthesized by methods known in the art and as described herein. Details of reaction schemes for synthesizing APC have been described in U.S. Pat. Nos. 5,705,640; 5,756,817; 5,955,499; and 6,140,532, all incorporated herein by reference in their entirety.

### APC Salt Formulations

Any suitable dosage form comprising the APC salts may be used in the methods of the invention. In some embodiments, the dosage formulation comprises the APC salt (which is pharmaceutically acceptable) and a pharmaceutically acceptable carrier. In some embodiments, the dosage form is an oral dosage form, e.g., a tablet or a capsule, e.g., an immediate release dosage form.

In some embodiments, the dosage form is an immediate release tablet that releases at least 85%, e.g., at least 85%, 90%, 95%, 96%, 97%, 98%, or 99%, of the APC salt contained therein within a period of less than 15 minutes after administration of the tablet to a subject. See, for example, U.S. Pat. No. 10,195,151, incorporated herein by reference in its entirety.

Formulations of the APC salt, including immediate release formulations, may be processed into unit dosage forms suitable for oral administration, such as for example, filled capsules, compressed tablets or caplets, or other dosage form suitable for oral administration using conventional techniques. Immediate release dosage forms prepared as described may be adapted for oral administration, so as to attain and maintain a therapeutic level of the compound over a preselected interval. In certain embodiments, an immediate release dosage form as described herein may comprise a solid oral dosage form of any desired shape and size including round, oval, oblong cylindrical, or polygonal. In one such embodiment, the surfaces of the immediate release dosage form may be flat, round, concave, or convex. In some embodiments, the shape may be selected to maximize surface area, e.g., to increase the rate of dissolution of the dosage form.

In particular, when the immediate release formulations are prepared as a tablet, the immediate release tablets may contain a relatively large percentage and absolute amount of the compound and so may be expected to improve patient compliance and convenience by replacing the need to ingest large amounts of liquids or liquid/solid suspensions. One or more immediate release tablets as described herein can be administered, by oral ingestion, e.g., closely spaced, in order to provide a therapeutically effective dose of the compound to the subject in a relatively short period of time.

Where desired or necessary, the outer surface of an immediate release dosage form may be coated, e.g., with a color coat or with a moisture barrier layer using materials and methods known in the art.

In some embodiments, the dosage formulation is an immediate release compressed tablet, the tablet comprising: the APC salt thereof in an amount of about 90-98% by weight of the tablet; at least one binder in an amount of about 1-5% by weight of the tablet; and at least one lubricant in an amount of about 0.1-2% by weight of the tablet; wherein the tablet releases at least 85% of the APC or a pharmaceutically acceptable salt thereof contained therein within a period of less than 15 minutes after administration of the tablet to a subject.

In one embodiment, the tablet comprises: the APC salt thereof in an amount of about 91-95% by weight of the tablet; at least one binder in an amount of about 2-3% by weight of the tablet; at least one lubricant in an amount of about 0.1-1% by weight of the tablet; and optionally, a cosmetic film coat in an amount of about 3-4% by weight of the tablet; wherein the tablet releases at least 85% of the APC or a pharmaceutically acceptable salt thereof contained therein within a period of less than 15 minutes after administration of the tablet to a subject.

In one embodiment, the tablet comprises: the APC salt thereof in an amount of about 93.22% by weight of the tablet; at least one binder (e.g., hydroxypropylcellulose) in an amount of about 2.87% by weight of the tablet; at least one lubricant (e.g., magnesium stearate) in an amount of about 0.52% by weight of the tablet; and optionally, a cosmetic film coat (e.g., Opadry®, for example, Opadry® II yellow) in an amount of about 3-4% by weight of the tablet; wherein the tablet releases at least 85% of the APC salt thereof contained therein within a period of less than 15 minutes after administration of the tablet to a subject.

In some embodiments, the composition is an immediate release oral dosage form of an APC salt, the oral dosage form comprising: the APC salt thereof in an amount of about 90-98% by weight of the oral dosage form; at least one binder in an amount of about 1-5% by weight of the oral dosage form; and at least one lubricant in an amount of about 0.1-2% by weight of the oral dosage form; wherein the oral dosage form releases at least 85% of the APC salt thereof contained therein within a period of less than 15 minutes after administration of the oral dosage form to a subject.

In certain embodiments, the tablet does not comprise a disintegrant. The term “disintegrant,” as used herein, refers to an agent added to a tablet to promote the breakup of the tablet in an aqueous environment. The tablets of the present invention are advantageous in that they dissolve rather than disintegrate. In the present invention the presence of disintegrant in the formulation may actually slow down release of APC.

In certain embodiments, the APC salt is present in an amount of about 90%, 90.5%, 91%, 91.5%, 92%, 92.5%, 93%, 93.5%, 94%, 94.5%, 95%, 95.5%, 96%, 96.5%, 97%, 97.5%, or 98% by weight of the tablet or any value or range therein. In certain embodiments, the APC salt thereof is present in an amount of about 90% to about 98%, about 92% to about 98%, about 94% to about 98%, about 96% to about 98%, about 90% to about 92%, about 90% to about 94%, about 90% to about 96%, about 92% to about 94%, about 92% to about 96%, or about 94% to about 96%.

In certain embodiments, the at least one binder is present in an amount of about 1%, 1.5%, 2%, 2.5%, 3%, 3.5%, 4%, 4.5%, or 5% by weight of the tablet or any value or range therein. In certain embodiments, the at least one binder is present in an amount of about 1% to about 5%, about 2% to about 5%, about 3% to about 5%, about 4% to about 5%, about 1% to about 2%, about 1% to about 3%, about 1% to about 4%, about 2% to about 3%, about 2% to about 4%, or about 3% to about 4%. The tablet may comprise at least one binder, e.g., 1, 2, 3, 4, 5, or more binders.

In certain embodiments, the at least one binder is selected from at least one of hydroxypropyl cellulose, ethylcellulose, hydroxypropyl methylcellulose, polyvinyl alcohol, hydroxyethyl cellulose, povidone, copovidone, pregelatinized starch, dextrin, gelatin, maltodextrin, zein, acacia, alginic acid, carbomers (cross-linked polyacrylates), polymethacrylates, sodium carboxymethylcellulose, guar gum, hydrogenated vegetable oil (type 1), methylcellulose, magnesium aluminum silicate, and sodium alginate or any combination thereof. In some embodiments, the at least one binder is hydroxypropyl cellulose.

In certain embodiments, the at least one lubricant is present in an amount of about 0.1%, 0.2%, 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% by weight of the tablet or any value or range therein. In certain embodiments, the at least one lubricant is present in an amount of about 0.1% to about 2.0%, about 0.5% to about 2.0%, about 1.0% to about 2.0%, about 1.5% to about 2.0%, about 0.1% to about 0.5%, about 0.1% to about 1.0%, about 0.1% to about 1.5%, about 0.5% to about 1.0%, about 0.5% to about 1.5%, or about 1.0% to about 1.5%. The tablet may comprise at least one lubricant, e.g., 1, 2, 3, 4, 5, or more lubricants. Where the immediate release formulation is provided as a tableted dosage form, still lower lubricant levels may be achieved with use of a “puffer” system during tableting. Such systems are known in the art, commercially available and apply lubricant directly to the punch and die surfaces rather than throughout the formulation.

In certain embodiments, the at least one lubricant is selected from at least one of magnesium stearate, stearic acid, calcium stearate, hydrogenated castor oil, hydrogenated vegetable oil, light mineral oil, magnesium stearate, mineral oil, polyethylene glycol, sodium benzoate, sodium stearyl fumarate, and zinc stearate or any combination thereof. In some embodiments, the at least one lubricant is magnesium stearate. In other embodiments, magnesium stearate may be used in combination with one or more other lubricants or a surfactant, such as sodium lauryl sulfate. In particular, if needed to overcome potential hydrophobic properties of magnesium stearate, sodium lauryl sulfate may also be included when using magnesium stearate (Remington: the Science and Practice of Pharmacy, 20^{th} edition, Gennaro, Ed., Lippincott Williams & Wilkins (2000)).

In some embodiments, the at least one binder is hydroxypropyl cellulose. In some embodiments, the at least one lubricant is magnesium stearate. In some embodiments, the at least one binder is hydroxypropyl cellulose and the at least one lubricant is magnesium stearate.

In certain embodiments, the tablet is coated. The coating may be, without limitation, a color overcoat.

The tablet may be any shape that is suitable for immediate release and allows the release of at least 85% of the APC salt contained therein within a period of less than 15 minutes after administration of the tablet to a subject. In some embodiments, the tablet maximizes surface area to volume ratio to promote rapid dissolution. In some embodiments, the tablet is oblong in shape.

The tablet may contain any amount of the APC salt suitable for administration as a unit dosage form. In some embodiments, the tablet contains the equivalent of about 1 mg to about 1000 mg of APC or any range or value therein, e.g., about 100 mg to about 500 mg, e.g., about 37.5 mg, about 75 mg, about 150 mg, or about 300 mg.

“Immediate release” as used herein, refers to a composition that releases the APC salt substantially completely into the gastrointestinal tract of the user within a period of less than about 15 minutes, usually between about 1 minute and about 15 minutes from ingestion. Such a delivery rate allows the drug to be absorbed by the gastrointestinal tract in a manner that is bioequivalent to an oral solution. Such rapid absorption will typically occur for an immediate release unit dosage form, such as a tablet, caplet or capsule, if the drug included in such dosage form dissolves in the upper portion the gastrointestinal tract.

Release rates can be measured using standard dissolution test methods. For example, the standard conditions may be those described in FDA guidance (e.g., 50 rpm, 37° C., USP 2 paddles, pH 1.2 and pH 6.8 media, 900 ml, 1 test article per vessel).

Immediate release formulations suitable for oral administration may comprise unit dosage forms, such as tablets, caplets or filled capsules, which can deliver a therapeutically effective dose of the APC salt upon ingestion thereof by the patient of one or more of said dosage forms, each of which can provide a dosage of, for example, about 37.5 mg to about 75 mg, or 75 mg to about 150 mg of APC. Additionally, the immediate release dosage forms can be shaped or scored to facilitate dose adjustment through tablet splitting. For example, a 75 mg tablet or caplet may be scored to facilitate tablet splitting into two 37.5 mg doses.

The formulation and structure of an immediate release dosage form as disclosed herein can be adjusted to provide immediate release performance that suits a particular dosing need. In particular, the formulation and structure of the dosage forms as described herein can be adjusted to provide any combination of the immediate release performance characteristics described herein. In particular embodiments, for example, an immediate release dosage form as disclosed herein provides rapid onset of action, releasing more than about 85%, such as, for example, more than about 90% or 95%, of the drug contained therein within a period of time selected from less than 15 minutes, less than 12 minutes, less than 10 minutes, and less than 5 minutes after administration.

Moreover, the rate of drug release from an immediate release dosage form as disclosed herein may be adjusted as needed to facilitate a desired dosing regimen or achieve targeted dosing. In certain such embodiments, the total amount of the APC salt in the dosage formulation may include an equivalent dose of about 10 mg to about 300 mg APC, about 30 mg to about 300 mg APC, about 100 mg to about 300 mg APC, or about 150 mg to about 300 mg APC, about 75 to 150 mg APC, about 37.5 to about 75 mg APC, and about 37.5 to about 150 mg APC. In particular embodiments, the equivalent dose of APC in the dosage formulation is 37.5 mg, and in other particular embodiments, the equivalent dose of APC in the dosage formulation is 75 mg. In some cases, such dosage formulations may be formed (e.g., scoring) to facilitate creating more than one dose from a particular dosage form.

The immediate release formulations provided herein generally include the APC salt and some level of lubricant to facilitate processing of the formulations into a unit dosage form. In some embodiments, therefore, the formulations described herein include a combination of the APC salt and lubricant, as described herein, and in certain such embodiments, the immediate release formulations are substantially free of other excipients or adjuvants. In other embodiments, the immediate release formulations described herein include a combination of the APC salt, lubricant, and binder, as described herein, and in certain such embodiments, the immediate release formulations are substantially free of other excipients or adjuvants. Though the immediate release formulations described herein may be formulated using a combination of drug and one or more of a lubricant and binder, in certain embodiments, the compositions described herein may include one or more additional excipients selected from, for example, fillers, compression aids, diluents, disintegrants, colorants, flavorants, buffering agents, coatings, glidants, or other suitable excipients.

The immediate release formulations described herein may be manufactured using standard techniques, such as wet granulation, roller compaction, fluid bed granulation, and dry powder blending. Suitable methods for the manufacture of the immediate release formulations and unit dosage forms described herein are provided, for example, in Remington, 20^{th} edition, Chapter 45 (Oral Solid Dosage Forms). It has been found that, even without the aid of binders or non-lubricating excipients, such as compression aids, wet granulation techniques can afford flowable granules with compression characteristics suitable for forming unit dosage forms as described herein. Therefore, in certain embodiments, where a drug content greater than about 85%, 90% or 95% by weight is desired for the immediate release formulation, wet granulation techniques may be used to prepare immediate release formulations as described herein. In such embodiments, conventional organic or aqueous solvents may be used in the wet granulation process. Suitable wet granulation processes can be performed as fluidized bed, high shear, or low shear (wet massing) granulation techniques, as are known in the art.

In addition to one or more the APC salt, lubricant, and binder, where desired, the immediate release formulations described herein may also include fillers or compression aids selected from at least one of lactose, calcium carbonate, calcium sulfate, compressible sugars, dextrates, dextrin, dextrose, kaolin, magnesium carbonate, magnesium oxide, maltodextrin, mannitol, microcrystalline cellulose, powdered cellulose, and sucrose. Where a filler or compression aid is used, in certain embodiments, it may be included in the immediate release formulation in an amount ranging from about 1%-15% by weight.

Where desired or necessary, the outer surface of an immediate release dosage form as disclosed herein may be coated with a moisture barrier layer using materials and methods known in the art. For example, where the APC salt delivered by the unit dosage form is highly hygroscopic, providing a moisture barrier layer over the immediate release dosage form as disclosed herein may be desirable. For example, protection of an immediate release dosage form as disclosed herein from water during storage may be provided or enhanced by coating the tablet with a coating of a substantially water soluble or insoluble polymer. Useful water-insoluble or water-resistant coating polymers include ethyl cellulose and polyvinyl acetates. Further water-insoluble or water resistant coating polymers include polyacrylates, polymethacrylates or the like. Suitable water-soluble polymers include polyvinyl alcohol and HPMC. Further suitable water-soluble polymers include PVP, HPC, HPEC, PEG, HEC and the like.

Where desired or necessary, the outer surface of an immediate release dosage form as disclosed herein may be coated with a color overcoat or other aesthetic or functional layer using materials and methods known in the art.

The dosage forms disclosed herein can also be provided as a kit comprising, separately packaged, a container comprising a plurality of immediate release tablets, which tablets can be individually packaged, as in foil envelopes or in a blister pack. The tablets can be packaged in many conformations with or without desiccants or other materials to prevent ingress of water. Instruction materials or means, such as printed labeling, can also be included for their administration, e.g., sequentially over a preselected time period and/or at preselected intervals, to yield the desired levels of APC in vivo for preselected periods of time, to treat a preselected condition.

### Subjects

The present invention finds use in research as well as veterinary and medical applications. Suitable subjects are generally mammalian subjects. The term “mammal” as used herein includes, but is not limited to, humans, non-human primates, cattle, sheep, goats, pigs, horses, cats, dog, rabbits, rodents (e.g., rats or mice), etc. Human subjects include neonates, infants, juveniles, adults and geriatric subjects.

In particular embodiments, the subject is a human subject that has excessive daytime sleepiness or another disorder amenable to treatment with the APC salt. In other embodiments, the subject used in the methods of the invention is an animal model of excessive daytime sleepiness or another disorder amenable to treatment with APC.

The subject can be a subject “in need of” the methods of the present invention, e.g., in need of the therapeutic effects of the inventive methods. For example, the subject can be a subject that is in need of improving on-the-road driving performance, e.g., lane drifts, reduced standard deviation of lateral position. In some embodiments, the subject may be experiencing excessive daytime sleepiness from narcolepsy or obstructive sleep apnea, or another disorder amenable to treatment with APC such as depression, is suspected of having excessive daytime sleepiness or another disorder amenable to treatment with APC, and/or is anticipated to experience excessive daytime sleepiness or another disorder amenable to treatment with APC, and experiences reduced on-the-road driving performance or is in need of improved on-the road driving performance or experiences excessive daytime sleepiness and is a motorist. In some embodiments, the subject is a human driver, i.e., motorist, and the methods and compositions of the invention are used for therapeutic and/or prophylactic treatment.

Having described the present invention, the same will be explained in greater detail in the following examples, which are included herein for illustration purposes only, and which are not intended to be limiting to the invention. Each of the examples has a self-contained list of references.

### EXAMPLES

### Example 1. Solriamfetol Impact on On-the-Road Driving Performance in Subjects with Narcolepsy

A randomized, double-blind, placebo-controlled, 2-period crossover study of solriamfetol in participants with narcolepsy. As few randomized controlled trials have evaluated on-the-road driving performance in this population, this study was conducted to evaluate the effects of solriamfetol on on-the-road driving performance in participants with narcolepsy.

Treatment periods consisted of 7 days of placebo or 7 days of solriamfetol (150 mg/day for 3 days, then 300 mg/day for 4 days); there was no washout between periods. This study was initiated before regulatory approval of solriamfetol or dosing recommendations were finalized; therefore, the 300-mg/day dose used here was based on prior phase 2 studies (Bogan et al., 2015; Ruoff et al., 2016) and is consistent with the maximum dose used in phase 3 trials of solriamfetol (Malhotra et al., 2020; Thorpy et al., 2019).

In this randomized, double-blind, placebo-controlled, crossover study, driving performance during a 1-hour on-road driving test was assessed at 2 hours and 6 hours post-dose following 7 days of treatment with solriamfetol (150 mg/day for 3 days, followed by 300 mg/day for 4 days) or placebo. The primary endpoint was standard deviation of lateral position (SDLP) at 2 hours post-dose.

The study included 24 participants with 54% male subjects, and a mean age of 40 years. 22 participants had evaluable SDLP data. At 2 hours post-dose, median SDLP was significantly lower (i.e., improved) with solriamfetol compared with placebo (19.08 vs. 20.46 cm [median difference, −1.9 cm], p=0.002). Four participants on solriamfetol and 7 on placebo had incomplete driving tests. At 6 hours post-dose, median SDLP was not statistically significantly different with solriamfetol compared with placebo (19.59 vs. 19.78 cm [median difference, −1.1 cm], p=0.125). Three participants on solriamfetol and 10 on placebo had incomplete driving tests. Common adverse events (≥5%) included headache, decreased appetite, and somnolence. Solriamfetol 300 mg/day improved on-the-road driving performance, at 2 hours post-administration in participants with narcolepsy.

### Results

A total of 29 participants were screened; of these, 4 failed screening and 25 were enrolled. One participant withdrew consent prior to dosing; therefore 24 participants comprised the safety population. Two participants withdrew from the study and did not have evaluable SDLP data at 2 hours post-dose (1 participant on placebo withdrew consent, and 1 participant on placebo withdrew due to adverse events of nausea and vomiting); therefore, the mITT population comprised 22 participants, all of whom completed the study.

The safety population was 54% male, with a mean age of 40.4 years. The observed mean (SD) SDLP at 2 hours post-dose was 20.9 (3.6) cm with placebo and 19.0 (3.6) cm with solriamfetol (mean [SD] difference, −1.91 [2.5] cm) and at 6 hours post-dose was 21.6 (5.8) cm and 19.8 (3.5) cm, respectively (mean [SD] difference, −1.62 [4.4] cm).

On the primary endpoint of SDLP at 2 hours post-dose, the median SDLP was significantly lower with solriamfetol compared with placebo (median difference, −1.90 cm [range, −6.7 to 2.6]; p=0.002); the median difference in SDLP at 6 hours post-dose was −1.1 cm (range, −12.1 to 6.0; p=0.125) (Table 1).

TABLE 1
Analysis of standard deviation of lateral position

| | Standard deviation of lateral position | | | | | |
|---|---|---|---|---|---|---|
| | Placebo | | Solriamfetol | | Median difference^{a} | |
| Time point | n | Median, cm | n | Median, cm | (range), cm | p^{b} |
| 2 hours post-dose^{c} | 22 | 20.46 | 22 | 19.08 | −1.9 (−6.7 to 2.6) | 0.002 |
| 6 hours post-dose | 21 | 19.78 | 22 | 19.59 | −1.1 (−12.1 to 6.0) | 0.125 |

ªSolriamfetol - placebo;
^{b}Wilcoxon rank sum test;
^{c}Primary endpoint.

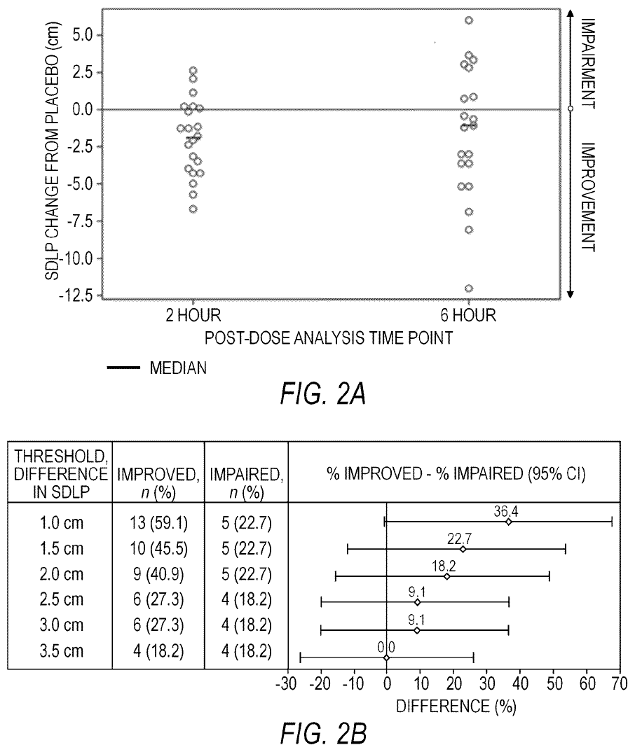

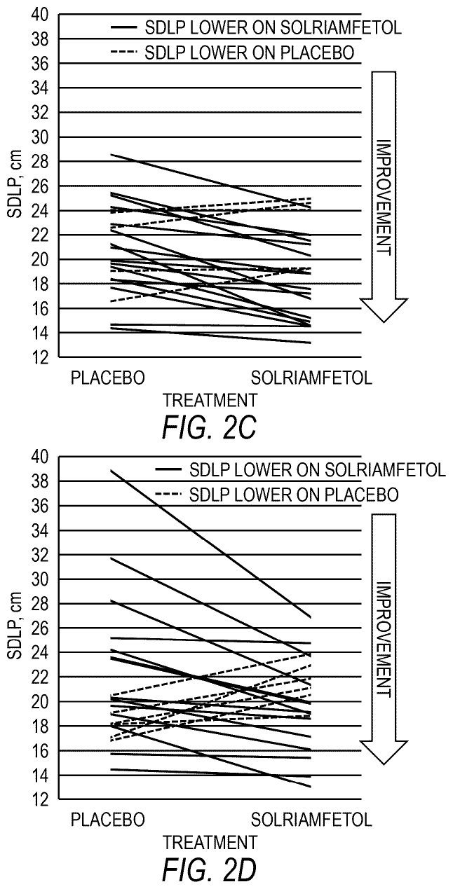

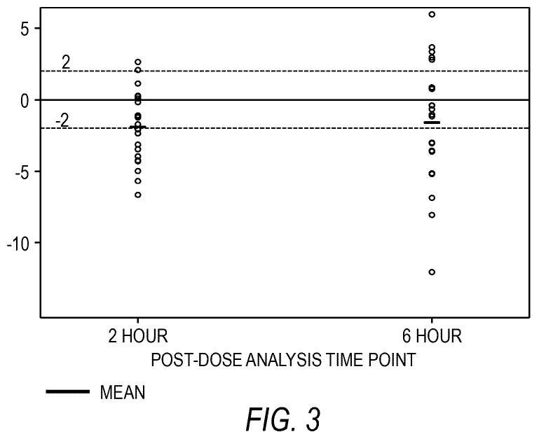

SDLP differences from placebo for individual participants' data are illustrated in FIG. 2A.

A total of 12 participants had ≥1 incomplete driving test. The number of incomplete driving tests was greater with placebo compared with solriamfetol at both 2 hours post-dose and 6 hours post-dose. Specifically, 11 participants had ≥1 incomplete test while on placebo (6 on both tests and 5 on a single test [1 at 2 hours; 4 at 6 hours]) and 5 participants had ≥1 incomplete test while on solriamfetol (2 on both tests and 3 on a single test [2 at 2 hours; 1 at 6 hours]). For both placebo and solriamfetol, at 2 hours post-dose, more tests were stopped by the instructor than by the participant; at 6 hours post-dose, more tests were stopped by the participant.

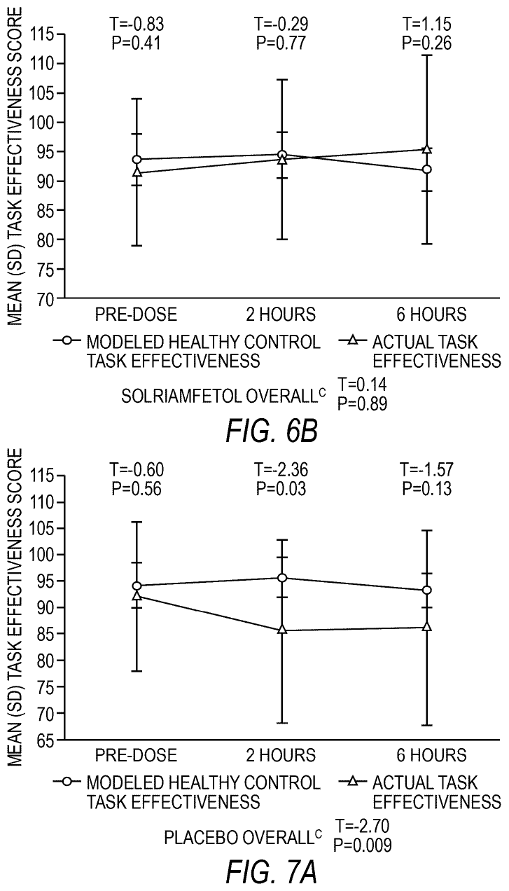

Overall higher percentages of participants had improvement (vs. impairment) on solriamfetol at all thresholds (from 1.0 to 3.5 cm, except 3.5 cm at 2 hours); however, single McNemar tests at each threshold did not demonstrate differences at either time point (all p≥0.05), and the maximum McNemar test did not show asymmetry at either 2 hours (FIG. 2B) or 6 hours post-dose (data not shown). Individual participant data for SDLP by treatment at 2 hours post-dose and 6 hours post-dose are illustrated in FIG. 2C and FIG. 2D, respectively.

On the additional secondary endpoints of standard deviation of speed and number of lane drifts, no differences were observed between solriamfetol and placebo at 2 or 6 hours post-dose; however, THAT scores, which measure perceived alertness over the preceding week (i.e., throughout the treatment period), were higher (indicating greater alertness) with solriamfetol compared with placebo. The least squares (LS) mean (standard error [SE]) standard deviation of speed at 2 hours post-dose was 2.8 (0.2) km/h with solriamfetol and 3.0 (0.2) km/h with placebo (LS mean difference, −0.22 [95% CI: −0.48, 0.05]) and at 6 hours was 3.1 (0.2) with solriamfetol and 3.2 (0.2) with placebo (LS mean difference, −0.11 [95% CI: −0.38, 0.17]). The LS mean (SE) number of lane drifts at 2 hours was 2.3 (0.8) with solriamfetol and 3.3 (0.8) with placebo (LS mean difference, −0.98 [95% CI: −3.1, 1.1]) and at 6 hours post-dose was 3.6 (0.8) with solriamfetol and 3.7 (0.8) with placebo (LS mean difference, −0.08 [95% CI: −2.2, 2.0]). The LS mean (SE) THAT score with placebo was 26.8 (1.4) and with solriamfetol was 34.0 (1.4), and the LS mean difference between solriamfetol and placebo was 7.1 (95% CI: 4.1, 10.2).

Treatment-emergent AEs (TEAEs) were reported for 20 (83%) participants; 6 (26%) participants experienced a TEAE while on placebo and 17 (74%) while on solriamfetol. One participant discontinued due to AEs (nausea and vomiting, which occurred while on placebo). All TEAEs were mild or moderate in severity. The most common TEAEs reported while participants were taking solriamfetol were headache and decreased appetite (n=4 each). There were no serious or fatal TEAEs. Changes from baseline in systolic and diastolic blood pressure and pulse rate were generally small, and their occurrence was proportionately similar between the 2 treatment groups and across treatment periods/visits (data not shown).

### Discussion

This study demonstrates that solriamfetol treatment at 150 mg/day for 3 days followed by 300 mg/day for 4 days significantly improved driving performance compared with placebo in participants with narcolepsy, as determined by the primary endpoint of SDLP at 2 hours post-dose. SDLP at 6 hours post-dose reflected some improvement with solriamfetol, although to a lesser extent.

While clear thresholds for clinically relevant improvement in SDLP have not been established, the clinical meaningfulness of the primary finding may be considered in the context of normative data. In an analysis of data from 74 healthy participants, the mean (SE) SDLP was 18.19 (0.46) cm with an upper limit of the 2-sided 95% CI of 19.09 cm (Vinckenbosch et al., 2021). The mean and median SDLP with placebo at 2 hours post-dose in the present study (20.88 and 20.46, respectively) exceeded this threshold, suggesting impairment in this population while on placebo, whereas the mean and median SDLP with solriamfetol (18.97 and 19.08, respectively) was within the CI of the aforementioned population of healthy participants, suggesting weaving and road-tracking ability within a healthy population norm while treated with solriamfetol.

Although this study was not designed to directly assess the risk of traffic accidents, studies of the effects of blood alcohol concentration and use of benzodiazepines on driving performance suggest that change in SDLP and crash risk are highly correlated and that SDLP is a valid predictor of alcohol- or drug-induced crash risk (Owens & Ramaekers, 2009). Data are lacking to confirm the predictive validity of SDLP in the context of wake-promoting agents and the potential for reducing the risk for traffic accidents. However, epidemiologic studies suggest stimulant and modafinil use reduces crash risk in patients with narcolepsy (Pizza et al., 2015; Tzeng et al., 2019). Nonetheless, the on-road driving test is generally regarded as the gold standard for assessing drug-induced changes in driving performance (Jongen et al., 2017).

Few studies of narcolepsy treatments have evaluated functional outcomes such as driving. In particular, studies of the effects of wake-promoting agents on measures of on-the-road driving performance, and specifically SDLP, in patients with narcolepsy are limited (Philip et al., 2014; Sagaspe et al., 2019). In a study of modafinil in patients with narcolepsy or idiopathic hypersomnia, the reduction in mean SDLP in an on-road driving test (conducted ~1.5 hours post-dose) with modafinil (400 mg/day) compared with placebo was not statistically significant (23.6±0.6 vs. 24.9±0.9 cm; p=0.06) (Philip et al., 2014). This is in contrast to the findings of this study, which showed a statistically significant improvement in SDLP at 2 hours post-dose with solriamfetol.

Several participants were unable to complete one or more driving tests, which could result in an underestimation of SDLP. The greater number of incomplete driving tests with placebo, particularly at the 6-hour post-dose time point, suggests that participants had less driving difficulty while on solriamfetol treatment. This finding supports the primary endpoint as it also reflects improvement in driving performance with solriamfetol. Considering these findings in the context of data from healthy participants, the overall percentage of driving tests stopped in this study was ~28% ( 24/87), which is nearly 9 times higher than in previous studies with healthy volunteers (3.1%) (Verster & Roth, 2012). In this study, 40% ( 17/43) of tests on placebo and 16% ( 7/44) on solriamfetol were stopped, whereas less than 1% and ~4% of the driving tests in unmedicated healthy volunteers and patients on various potentially sedating drug treatments, respectively, were stopped in previous studies (Verster & Roth, 2012). No participants stopped driving tests in the aforementioned modafinil study (Philip et al., 2014), despite the fact that those tests covered more than twice the distance, though participants in that study were allowed to remain on anticataleptic medication in contrast to the current study. This shows that, with and without medication, a significant percentage of participants in the current study had problems maintaining alertness for up to an hour during prolonged highway driving. Interestingly, more tests were stopped by the participant than by the instructor (13 vs. 11). In contrast, in studies with healthy volunteers the decision to stop was 3 to 4 times more often made by the instructor than by the participant (Verster & Roth, 2012). This suggests that participants with narcolepsy in this study seemed aware of potential impairment and were careful to avoid further risks. If participants decided to stop before effects on SDLP were detectable, the observed treatment effect on SDLP may be an underestimation of the ability of solriamfetol to improve performance in this setting. For example, if participants had not stopped their tests while on placebo, SDLP likely would have reflected greater impairment.

The SD of SDLP has been reported to range from 2.6 to 4.2 cm in healthy participants or in participants with ADHD with or without stimulant or hypnotic treatment (Vermeeren et al., 2014; Verster et al., 2008; Verster & Roth, 2011). The power calculation performed to determine the sample size required for the present study therefore assumed an SD of 3.0 cm, in line with the estimated SD for power estimation in a study of methylphenidate use in participants with attention deficit hyperactivity disorder (Verster et al., 2008). However, the observed SD of SDLP in this study ranged from 3.5 to 5.8 cm, suggesting the study may have been underpowered to detect a difference in SDLP. Although an improvement was still detected at 2 hours post-dose in participants treated with solriamfetol, it was not maintained at 6 hours post-dose. These observations align with the pharmacokinetic profile of solriamfetol, which was demonstrated to have a median time to peak plasma concentration of 2 hours and a mean half-life of 5.9 hours in fasting conditions (3 hours and 6.1 hours, respectively, in fed conditions) (Zomorodi, Kankam, & Lu, 2019).

Other secondary driving outcomes (standard deviation of speed and lane drifts) showed minimal differences between solriamfetol and placebo, which may be due to a relative lack of sensitivity or statistical power. Standard deviation of speed is less sensitive to changes in driving performance parameters compared with SDLP (Irwin, Iudakhina, Desbrow, & McCartney, 2017; Verster & Roth, 2014). In contrast, the difference between treatments in THAT scores was more substantial and suggested greater alertness with solriamfetol. This improvement is consistent with the established wake-promoting effects of solriamfetol on other measures, such as the Epworth Sleepiness Scale and the Maintenance of Wakefulness Test, which showed treatment differences from placebo (least squares mean) of −2.2 to −4.7 points and 2.6 to 10.1 minutes, respectively, after 12 weeks of treatment with solriamfetol at doses of 75 to 300 mg/day in the phase 3 trial of solriamfetol in participants with narcolepsy (Malhotra et al., 2020; Thorpy et al., 2019). These wake-promoting effects have been shown to be maintained for up to 12 months in an open-label extension study (Malhotra et al., 2020).

The tolerability profile of solriamfetol in this study is consistent with those observed in other clinical trials in participants with narcolepsy (Ruoff et al., 2016; Thorpy et al., 2019). All TEAEs were mild or moderate in severity. No participant discontinued the study due to AEs while taking solriamfetol.

### Conclusion

Solriamfetol 300 mg/day significantly improved SDLP, an important measure of driving performance, in participants with narcolepsy at 2 hours post-dose, the primary efficacy outcome. The difference in SDLP at 6 hours post-dose, a secondary outcome, was not significant. However, these findings indicate that the robust wake-promoting efficacy of solriamfetol demonstrated in clinical trials resulted in improved real-world functional performance in participants with narcolepsy.

### Participants

Participants were recruited from sleep clinics or clinical sites. Eligible participants were men and women aged 21 to 75 years with a diagnosis of narcolepsy, per the International Classification of Sleep Disorders—Third Edition (American Academy of Sleep Medicine, 2014) or the Diagnostic and Statistical Manual of Mental Disorders—Fifth Edition (American Psychiatric Association, 2013). Other study inclusion criteria were average total nightly sleep≥6 hours (as verified through actigraphy and sleep diaries), body mass index (BMI) 18 to <40 kg/m², normal vision (corrected or uncorrected), possession of a valid driver's license for ≥1 year, history of driving on a regular basis, and ability to operate a vehicle with a manual transmission.

Key exclusion criteria included occupational nighttime shift work, usual bedtime after 1:00 A.M., clinically relevant medical or psychiatric disorders (other than narcolepsy) associated with EDS, history or presence of unstable medical or psychiatric conditions, pregnancy, previous use of solriamfetol, excessive caffeine use (>8 cups of coffee/day), or smoking>10 cigarettes/day. Use of medications that affect sleep-wake functions was prohibited during the study and required a washout period prior to the first dose of study treatment (stimulants or alerting agents, 3 days; sodium oxybate, 7 days; and other medications that could affect sleep-wake functions or monoamine oxidase inhibitors, 14 days or 5 half-lives).

### Study Design

This was a randomized, double-blind, placebo-controlled, 2-period crossover study of solriamfetol in participants with narcolepsy. Treatment periods consisted of 7 days of placebo or 7 days of solriamfetol (150 mg/day for 3 days, then 300 mg/day for 4 days); there was no washout between periods. This study was initiated before regulatory approval of solriamfetol or dosing recommendations were finalized; therefore, the 300-mg/day dose used here was based on prior phase 2 studies (Bogan et al., 2015; Ruoff et al., 2016) and is consistent with the maximum dose used in phase 3 trials of solriamfetol (Malhotra et al., 2020; Thorpy et al., 2019), although it exceeds the currently approved maximum dose.

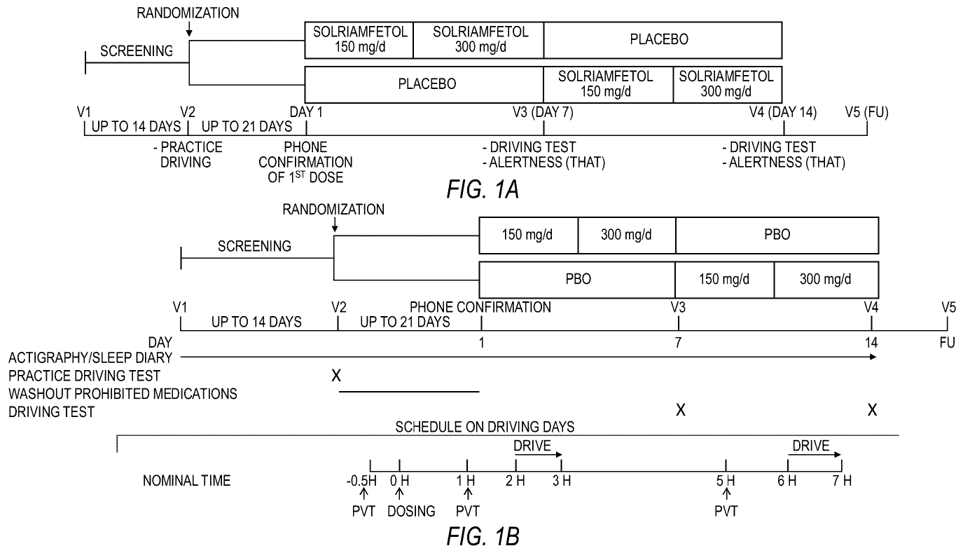

Eligible participants were randomly assigned 1:1 to one of 2 treatment sequences: solriamfetol followed by placebo (solriamfetol/placebo) or placebo followed by solriamfetol (placebo/solriamfetol) (FIG. 1A). Randomization was performed by the investigator with an interactive response technology system; assignment to one treatment sequence or the other followed a blocked randomization schedule generated by a statistician (not involved in the analysis of the study data) before the start of the study. Solriamfetol 150- and 300-mg tablets and placebo tablets were supplied in identical opaque gelatin capsules to ensure adequate blinding. All study personnel were blinded to study treatments.

### Procedures

The study included a screening/washout period of ≤5 weeks prior to the first dose of study treatment, during which eligibility was assessed (including general safety assessments), prohibited medications were washed out, and participants completed a practice driving test. Eligible participants were randomized and started taking study drug at home to ensure a steady state of 300 mg was achieved by driving test day. Participants were contacted by telephone 2 days prior to starting study treatment and on Day 1 of Period 1 to confirm their first dose of study treatment. On Day 7 and 14 (i.e., Day 7 of each period), visits were conducted to evaluate driving performance. A safety follow-up visit was conducted approximately 1 week after completion of Period 2.

On non-test days, participants were instructed to take a single capsule orally once daily, within 1 hour of waking in the morning, on an empty stomach, and then to wait ≥30 minutes before having breakfast. Timing of administration on non-test days was not as critical as timing of administration on driving test days, as long as the study drug was taken in compliance with label instructions (Sunosi™ (solriamfetol) tablets Prescribing Information, 2021; Sunosi™ (solriamfetol) tablets Summary of Product Characteristics, 2020). On driving test days, the capsule for that day was administered at the driving test site in the presence of an investigator at 8:45 A.M. (2 hours before the start of the first drive); 30 minutes after administration, participants received a light breakfast. Throughout the study, caffeine users were instructed to not increase their use during the study, and nicotine users were instructed to maintain a consistent level of use. In addition, on driving test days, 1 cup of black coffee was permitted prior to arrival at the test site, with no additional consumption until after the second driving test, and nicotine use was restricted to 1 cigarette on waking, with no other use until after the study procedures were completed on those days.

At the end of each treatment period, a standardized on-road driving test (Verster & Roth, 2011) was conducted at 2 hours and at 6 hours after administration of drug or placebo (FIG. 1A). For each test (~1 hour in duration), participants drove a specially instrumented vehicle over a 100 km (~62 miles) primary highway circuit; they were accompanied by a licensed driving instructor with access to dual controls (brakes, clutch, accelerator). Participants were instructed to maintain both a steady lateral position between the delineated boundaries of the slower (right) traffic lane and a constant speed of 95 km/h (~59 mph). Participants were permitted to deviate from these instructions only to pass a slower vehicle, to respond to slower traffic ahead, or to exit and reenter the highway at the turnaround point (these events were removed for the purpose of data analysis by 2 experienced editors of the driving data). Vehicle speed and lateral distance to the left-lane line were continuously recorded, and the data stored on an onboard computer. The driving test could be stopped by the participant or by the accompanying driving instructor if considered it unsafe to continue.

### Assessments and Outcomes

The primary outcome assessment from the driving tests was standard deviation of lateral position (SDLP) in centimeters—a measure of “weaving” or road-tracking control (Ramaekers, 2017; Verster & Roth, 2011). For participants who did not complete the driving test, SDLP data from the part of the test that was completed were analyzed, though this could have impacted the observed treatment effect on SDLP. Standard deviation of speed and number of lane drifts (defined as deviations>100 cm from the absolute lateral position within an 8-second window) were also determined from driving test data.

The Toronto Hospital Alertness Test (THAT) is a 10-item self-report questionnaire that measures perceived alertness over the previous week; scores can range from 0 to 50, with higher scores indicating greater alertness (Shapiro et al., 2006). This assessment was administered at the end of each 7-day treatment period to evaluate participants' perceived alertness throughout the treatment period. Participants completed the THAT prior to administration of study treatment at the visits on driving test days; this timing (i.e., with respect to dosing) is not expected to affect THAT scores, since the questionnaire does not measure alertness at a point in time, but over the preceding week.

Safety assessments included a physical examination, ECG, clinical laboratory tests, and assessment of adverse events (AEs).

### Statistical Analyses

The primary efficacy endpoint was SDLP at 2 hours post-dose; secondary efficacy endpoints included SDLP at 6 hours post-dose, percentages of participants with improved or impaired driving on solriamfetol compared with placebo, standard deviation of speed, lane drifts, and THAT score.

For the primary endpoint, the null hypothesis was that mean SDLP with solriamfetol and mean SDLP with placebo were equal; the alternative hypothesis was that they were not equal. The treatment difference in mean SDLP between solriamfetol and placebo at 2 hours post-dose was tested; a 5% type I error rate (p<0.05) was considered statistically significant. A sample size of 30 participants would provide 90% power to detect a mean difference of 2.0 cm on the primary outcome measure of SDLP (Ramaekers, Kuypers, & Samyn, 2006; Verster et al., 2008), assuming an SD of 3.0 cm (Verster et al., 2008) and a 2-sided 0.05 significance level using a paired t-test. To account for 10% dropouts without evaluable SDLP data, a sample size of 33 participants was planned. Post hoc calculations based on the number of enrolled participants indicated an estimated power of ~78%.

Efficacy analyses were performed with data from the modified intent-to-treat analysis population, which comprised all randomized participants who received ≥1 dose of study drug and had evaluable SDLP data at 2 hours post-dose.

Change in SDLP was analyzed with a repeated mixed-effects analysis of variance (ANOVA). Normality assumption was examined on the mixed effect model residuals using the Shapiro-Wilk normality test; it was observed that change in SDLP did not meet the normality assumption, and therefore the Wilcoxon signed rank test was used to compare the pairwise treatment differences.

Maximally selected McNemar symmetry analyses (Laska, Meisner, & Wanderling, 2012) were used to detect asymmetry in the distribution of the change in driving performance at 2 hours and 6 hours post-dose. Single McNemar tests were used to analyze the difference in proportions of participants with improved or impaired driving performance at relevant thresholds. Thresholds of 1.0, 1.5, 2.0, 2.5, 3.0, and 3.5 cm were used. In comparisons of solriamfetol and placebo, improvement was defined as a decrease in SDLP in participants treated with solriamfetol compared to placebo at the threshold, and impairment was defined as an increase in SDLP at the threshold or failure to complete the driving test while on solriamfetol because of sleepiness or safety concerns (regardless of their performance on placebo; participants who failed to complete the driving test while on placebo but who completed the test while on solriamfetol were not counted as impaired or improved).

The number of participants who failed to complete the driving test and the duration of the drive before stopping were summarized descriptively. Additional secondary efficacy measures (standard deviation of speed, number of lane drifts, and THAT scores) were analyzed using a similar ANOVA method as described for SDLP. No multiplicity adjustments were made in the efficacy analyses for multiple endpoints, and all p values are therefore nominal.

Demographic, narcolepsy history, and safety data were summarized for the safety population, which included all participants who received ≥1 dose of study drug. No formal statistical testing was performed on these parameters.

### Example 2. Solriamfetol Impact on On-the-Road Driving Performance in Subjects with Obstructive Sleep Apnea

This study was designed to assess the effects of solriamfetol on driving performance in subjects with OSA. In addition, it also assessed the effects of solriamfetol on measures of attention, reaction time (RT), and sustained attention and vigilance. In this study, the psychomotor vigilance test (PVT) was used to assess psychomotor performance, with errors of commission on the PVT used as a measure of impulsivity. A randomized, double-blind, placebo-controlled crossover study design was selected for this study as it allowed for an intra-subject comparison between solriamfetol and placebo in evaluating driving performance. During the solriamfetol treatment period, subjects received 150 mg/day for 3 days, followed by 300 mg/day for 4 days. The driving test was conducted after steady state of the 300 mg/day dose was reached on the seventh day of dosing.

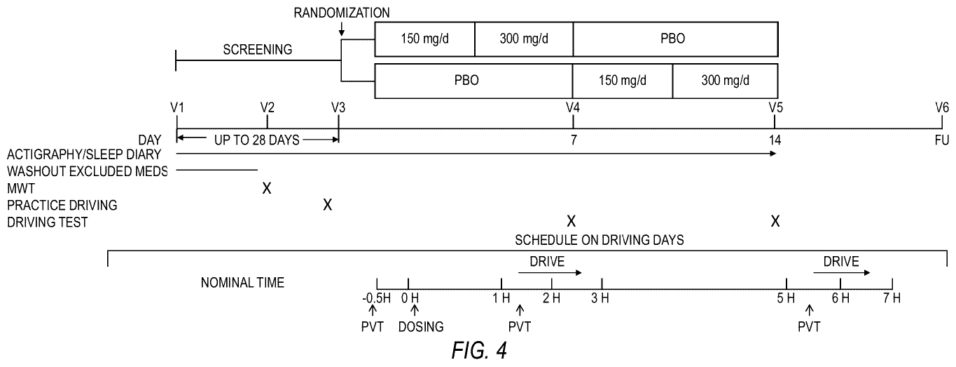

This was a randomized, double-blind, placebo-controlled, crossover, on-road driving study in subjects with EDS due to OSA. A total of 40 adult subjects were planned for enrollment, and 34 subjects were randomized. Subjects received either solriamfetol (150 mg QD for 3 days, followed by 300 mg QD for 4 days) or matching placebo for 7 days, and then crossed over to receive the other treatment regimen for 7 days (FIG. 4). On Day 7 of each treatment period (i.e., on Study Days 7 and 14), all randomized subjects had a study visit to undergo 2 driving performance tests, one at 2 hours (between 1 to 3 hours) and the other at 6 hours (between 5 to 7 hours) after the morning dose. The PVT was administered predose and prior to each driving test. Actigraphy and a sleep diary were used to assess daily sleep patterns. The Toronto Hospital Alertness Test (THAT) was administered at baseline and at the end of each treatment period. A Follow-up Visit was performed approximately 7 days after the final dose of study drug. Subjects were recruited at clinical sites. Eligibility was determined through screening procedures (including a Maintenance of Wakefulness Test [MWT]) at the clinical sites after the washout of prohibited medications, and a practice driving test at the driving test site. A total of 40 adult subjects were planned for enrollment; 34 subjects were randomized and analyzed for safety and efficacy.

### Efficacy Results

Primary efficacy endpoint of SDLP at 2 hours postdose was met and a key secondary efficacy endpoint of SDLP at 6 hours postdose was also met. Solriamfetol demonstrated a statistically significant reduction in SDLP at 2 hours and 6 hours postdose as compared with placebo, which demonstrated that solriamfetol improved this aspect of driving performance compared with placebo at 2 hours postdose (LS means: −1.084 cm, p=0.0062) and 6 hours postdose (LS means: −0.804 cm, p=0.0432) in the mITT population. Robustness of the findings was confirmed in the sensitivity analyses (Per Protocol [PP] population) for SDLP at both these time points (p=0.0055 and p=0.0181, respectively).

The primary outcome measure was the mean change in SDLP (vehicle “weaving”) at 2 hours postdose in the mITT population. The normality and homogeneity assumptions were tested according to the SAP and no deviation from the assumptions were detected.

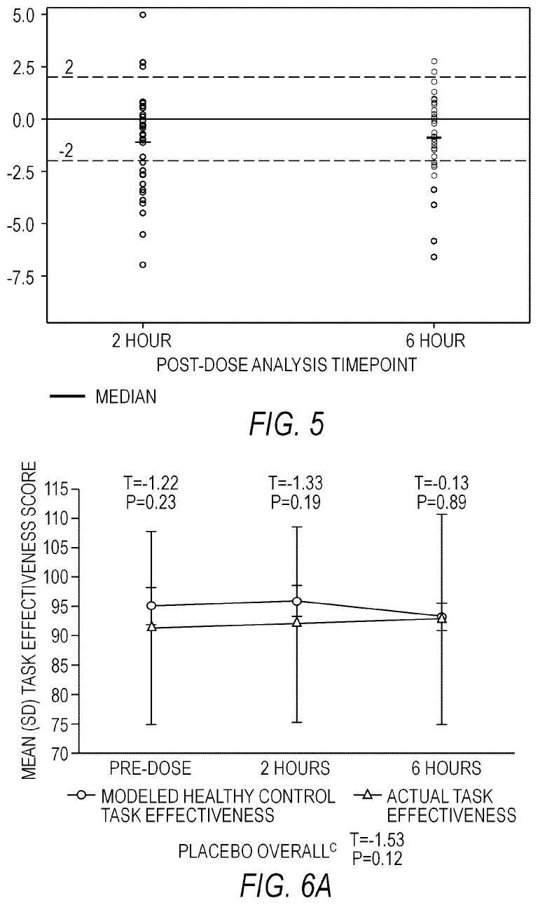

In the primary analysis, the SDLP at 2 hours postdose was statistically significant with a least squares (LS) mean difference between solriamfetol and placebo of −1.084, p=0.0062, suggesting that solriamfetol improves driving performance in OSA subjects at 2 hours postdose compared with placebo (Table 2 and FIG. 5).

TABLE 2
Standard Deviation of Lateral Position at 2 Hours PostDose: Summary
of Observed Values and Primary Analysis (mITT Population)

| | | | Difference |
|---|---|---|---|
| Standard Deviation of | | | (JZP-110 − |
| Lateral Position | Placebo | JZP-110 | Placebo) |
| (cm) | (N = 34) | (N = 34) | (N = 34) |
| Overall Summary | | | |
| n | 33 | 34 | 33 |
| Mean (SD) | 19.95 (4.181) | 18.83 (3.690) | −1.11 (2.520) |
| Median | 19.10 | 18.39 | −0.78 |
| Range | 13.5, 29.1 | 12.4, 25.9 | −7.0, 5.0 |
| Primary Analysis | | | |
| LS Mean (SE) | 19.92 (0.630) | 18.83 (0.627) | |
| LS Mean Difference | | −1.084 | |
| (JZP-110 − Placebo) | | | |
| 95% CI | | −1.848, −0.319 | |
| p-Value | | 0.0062 | |

ANOVA = analysis of variance; CI = confidence interval; LS = least square; mITT = modified Intent-to-Treat; n = number of subjects with non-missing value; SD = standard deviation; SDLP = standard deviation of lateral position; SE = standard error.

The primary outcome measure of mean change in SDLP was analyzed using a repeated mixed effect ANOVA model. The model included treatment (JZP-110 and placebo), driving performance test (2 hours post-dose and 6 hours post-dose), treatment period, and treatment by driving performance test interaction as fixed effects and subject as a random effect. The assumption of normal distribution of the data required for ANOVA model was examined using the Shapiro-Wilk Normality test on the residuals from the mixed-effect model or (P=0.3226). The homogeneity of variance between treatments was evaluated using the Levene test (P=0.4087).

Driving performance was also evaluated by number of driving lapses and by standard deviation of speed (SDS) at 2 hours and 6 hours postdose. At 2 hours postdose, subjects who received solriamfetol versus placebo had fewer mean lapses (Least Squares [LS] mean difference, −1.128 lapses; nominal p=0.0806); at 6 hours postdose the mean lapses were similar between the 2 treatment groups (LS mean difference, 0.050 lapses, nominal p=0.9391). At both timepoints, the LS mean difference in SDS between both treatment groups were similar (2 hours postdose: 0.068 km/h, nominal p=0.41116 and 6 hours postdose: −0.109 km/h, nominal p=0.1991).

Improvement in mean RT at 6 hours postdose was observed in subjects who received solriamfetol compared with placebo (LS mean difference, −54.494 msec; nominal p=0.0387) with a trend towards improvement for other vigilance parameters (1/RT, lapses and errors of commission). On the THAT, subjects had a higher LS mean total score when treated with solriamfetol than when treated with placebo (27.52 versus 23.94; nominal p=0.0241) in the mITT population. A similar trend was observed in the PP population (nominal p=0.0781).

### Secondary Efficacy Endpoints

### Standard Deviation of Lateral Position at 6 Hours Postdose

As observed for the primary endpoint, the mean SDLP at 6 hours postdose in the mITT population was greater in subjects who received placebo compared with solriamfetol with a LS mean (SD) difference of −0.804 cm which was statistically significant (p=0.0432; Table 3).

Sensitivity analysis for SDLP at 6 hours postdose was performed in the PP population and was statistically significant in favor of solriamfetol, supporting the robustness of the analysis. The LS mean (SD) difference was −1.037 cm, p=0.0181.

TABLE 3
Standard Deviation of Lateral Position at 6 Hours PostDose:
Summary of Observed Value Overall (mITT Population)

| | | |
|---|---|---|
| Standard Deviation of | Placebo | JZP-110 |
| Lateral Position (cm) | (N = 29) | (N = 29) |

Overall Summary

| | | |
|---|---|---|
| n | 32 | 32 |
| LS Mean (SE) | 20.04 (0.632) | 19.24 (0.631) |
| LS Mean Difference | | −0.804 |
| (JZP-110 − Placebo) | | |
| 95% CI | | −1.583, −0.025 |
| p-Value | | 0.0432 |

ANOVA = analysis of variance;
N = number of subjects within each treatment assigned;
n = number of subjects with non-missing value;
SDLP = standard deviation of lateral position

The primary outcome measure of mean change in SDLP will be analyzed using a repeated mixed effect ANOVA model. The model will include treatment (JZP-110 and placebo), driving performance test (2 hours post-dose and 6 hours post-dose), treatment period, and treatment by driving performance test interaction as fixed effects and subject as a random effect. The assumption of normal distribution of the data required for ANOVA model is examined using the Shapiro-Wilk Normality test on the residuals from the mixed-effect model (P=0.3226). The homogeneity of variance between treatments is evaluated using the Levene test (P=0.4087).

Standard Deviation of Lateral Position by Treatment Sequence (mITT Population)

When subjects received placebo as initial study treatment, they had lower mean SDLP values after receiving solriamfetol as compared with placebo at both 2 hours postdose (19.08 cm vs 20.99 cm) and 6 hours postdose (19.23 cm vs 21.07 cm). The difference in median values between the 2 treatments (solriamfetol-placebo) was −1.91 cmat both time points (Table 4). When subjects received solriamfetol as the initial study treatment, mean SDLP was similar on active drug vs placebo with a difference of −0.26 cm at 2 hours postdose and a difference of 0.20 cm at 6 hours postdose.

TABLE 4
Standard Deviation of Lateral Position Postdose: Summary of
Observed Values by Treatment Sequence (mITT Population)

| | | | Difference | |
|---|---|---|---|---|
| Standard Deviation of | | | (JZP-110/ | |
| Lateral Position (cm) | Placebo | JZP-110 | Placebo) | |
| 2 Hours Postdose | | | | |
| JZP-110/Placebo | | | | |
| n | 16 | 17 | 16 | |
| Mean (SD) | 18.85 (4.166) | 18.58 (3.831) | −0.26 | (2.694) |

| | | | |
|---|---|---|---|
| Median | 18.52 | 17.98 | −0.18 |
| Range | 13.6, 27.9 | 13.3, 25.9 | −7.0, 5.0 |

Placebo/JZP-110

| n | 17 | 17 | 17 | |
|---|---|---|---|---|
| Mean (SD) | 20.99 (4.042) | 19.08 (3.643) | −1.91 | (2.120) |
| Median | 21.70 | 20.00 | −2.46 | |
| Range | 13.5, 29.1 | 12.4, 23.8 | −5.5, 2.7 | |
| 6 Hours Postdose | | | | |
| JZP-110/Placebo | | | | |
| n | 15 | 16 | 15 | |
| Mean (SD) | 18.90 (3.367) | 19.23 (3.224) | 0.20 | (1.566) |
| Median | 17.89 | 18.92 | 0.43 | |
| Range | 13.6, 24.4 | 14.5, 25.4 | −2.3, 2.8 | |
| Placebo/JZP-110 | | | | |
| n | 17 | 16 | 16 | |
| Mean (SD) | 21.07 (3.639) | 19.23 (3.593) | −1.91 | (2.129) |

| | | | |
|---|---|---|---|
| Median | 21.68 | 19.24 | −1.33 |
| Range | 13.9, 27.0 | 13.0, 24.3 | −6.6, 1.0 |

mITT = modified Intent-to-Treat; n = number of subjects with non-missing value; SD = standard deviation.
Driving Performance: Standard Deviation of Speed (SDS) and Driving Lapses

Driving performance was also evaluated by number of driving lapses and by SDS. At 2 hours postdose, subjects who received solriamfetol versus placebo had fewer mean lapses; however, the LS mean difference between solriamfetol versus placebo was −1.128 lapses, p=nominal 0.0806. At 6 hours postdose, the number of lapses were similar for subjects who received solriamfetol versus placebo, with an LS mean difference of 0.050 lapses, p=nominal 0.9391. Similarly, at 2 hours and 6 hours postdose, the LS mean difference in SDS between solriamfetol and placebo were similar (0.068 km/h, nominal p=0.41116 and −0.109 km/h, p=nominal 0.1991, respectively). The sensitivity analysis performed with the PP population confirmed these findings.

McNemar Test of Proportion of Subjects with Improved or Impaired Driving

The Maximum McNemar test was performed to evaluate the proportion of subjects showing improved or impaired driving in the SDLP over all relevant thresholds (as described in Section 9.7.2.3). The result of the Maximum McNemar test did not show asymmetry in the distribution of the change in driving performance between solriamfetol and placebo at 2 hours and 6 hours post-dose (0.05<nominal p<0.1 at 2 hours; nominal p>0.1 at 6 hours; Table 5).

For the single McNemar test, the proportions of subjects with improved versus impaired driving at a threshold of 1.0 cm at 2 h post dose were: 29.4% improved drivers versus impaired, nominal p=0.0414. At all other threshholds (1.5 cm to 3.5 cm), the difference in proportion was numerically smaller at the 2 h postdose and 6 h postdose time points. Results were similar for the PP population.

Note that the number of subjects who failed to complete the driving test was greater for the placebo condition as compared with solriamfetol (7 subjects vs 3, respectively). Subjects were considered to be impaired if they failed to complete the driving test. However, subjects who failed to complete the test while in the placebo condition (regardless of treatment sequence) were not included among those who were considered impaired.

TABLE 5
Analysis: Maximally Selected McNemar Test of Proportion of Subjects
with Improved or Impaired Driving (mITT Population)

| Time Point | Threshold | p-value* |
|---|---|---|
| 2 Hours Post-dose | 0.83 | 0.05 < P < 0.1 |
| 6 Hours Post-dose | 5.83 | >0.1 |

mITT = modified intent-to-treat

The Maximally selected test statistic was compared with the Critical Values of Max McNemar Test for the sample size. The p-value range was one of these: P<0.01, P=0.01, 0.01<P<0.025, P=0.025, 0.025<P<0.05, P=0.05, 0.05<P<0.1, P=0.1, P>0.1. Threshold was the lower limit of the thresholds that could achieve the p-value. *p-values are nominal.

### Toronto Hospital Alertness Test

On the THAT, subjects who received solriamfetol had a higher median total score than those who received placebo: 28.5 vs 22.0, respectively, with a difference of 4.0 ( ). In the analysis, the LS mean difference between solriamfetol and placebo was 3.579, nominal p=0.0241; Table 6, suggesting that subjects who received solriamfetol had a greater perceived alertness than those who received placebo. Sensitivity analysis performed with the PP population, a similar trend was observed with an LS mean difference of 2.983 (nominal p=0.0781).

TABLE 6
Toronto Hospital Alertness Test: Summary of
Observed Value and Analysis (mITT Population)

| | Placebo | JZP-110 | |
|---|---|---|---|
| THAT Total Score | (N = 34) | (N = 34) | P-value |
| Total Score at End of Treatment Period | | | |
| n | 33 | 34 | |
| Mean (SD) | 24.0 (8.52) | 27.5 (7.60) | |
| Median | 22.0 | 28.5 | |
| Range | 10, 40 | 14, 43 | |
| Analysis | | | |
| n | 33 | 34 | |
| LS Mean (SE) | 23.94 (1.180) | 27.52 (1.162) | |
| LS Mean Difference | | 3.579 | |
| (JZP-110 − Placebo) | | | |
| 95% CI | | 0.500, 6.658 | |
| p-Value* | | | 0.0241 |

N = number of subjects within each treatment assigned. n = number of subjects with non-missing value.

THAT is analyzed using a mixed effect analysis of covariance (ANCOVA) model. The model includes baseline, treatment (JZP-110 and placebo), treatment period, treatment sequence as fixed effects and subject as a random effect. *p-value is nominal.

### Psychomotor Vigilance Test (PVT)

The PVT is a sustained-attention, reaction-timed task; subjects were instructed to respond to the appearance of a visual stimulus on a computer screen by pushing a response button as quickly as possible. The PVT measures included mean RT, 1/RT, lapses (RT>500 msec), and errors of commission.

Although a trend towards improvement in all vigilance parameters (mean RT, 1/RT, lapses and errors of commission) was observed for subjects who received solriamfetol versus placebo, at both 2 hours and 6 hours postdose, the nominal p-values were >0.05 for any of the parameters except for the mean RT (Table 19), which had improvement at the 6 hours postdose time point for subjects who received solriamfetol versus placebo with a LS mean difference of −54.494 msec, nominal p=0.0387.

### Actigraphy and Sleep Diary

Subjects had actigraphy measurements to record their sleep/wake patterns continuously over 24 hours, and used sleep diaries to ensure they had adequate sleep and maintained a consistent sleep schedule during the study.

For actigraphy assessments, the mean change from baseline to the end of the treatment period was noted for each of 5 parameters. In total sleep time (average total actual minutes of sleep obtained within a day, from noon to noon), subjects on placebo experienced an increase in total time of 22.34 minutes, while subjects on solriamfetol experienced a reduction of 16.29 minutes (Table 7). The average total awake time between initial sleep onset and final awakening from all sleep episodes within a day (WASO) decreased for subjects who received either placebo (−1.98 minutes) or solriamfetol (−0.69 minutes). The average change in sleep efficiency ([total sleep time/time in bed]*100 from all sleep episodes within a day) was 1.94% during placebo treatment and −0.04% while receiving active drug. The differences between the 2 treatments in total sleep time and WASO was not considered clinically meaningful. The differences between the 2 treatments in total sleep time and WASO was not considered clinically meaningful. The method of data compilation did not allow the differentiation of sleep time into daytime or nighttime sleep. The relatively low total number of subjects reporting insomnia on solriamfetol suggest that these nonclinically meaningful differences may in part be due to the variance in wakefulness during the day as mean sleep efficiency (i.e., nighttime sleeping patterns) did not differ greatly between the 2 treatments.

TABLE 7
Actigraphy and Sleep Diary: Summary of Change from Baseline (mITT Population)

| | | | Difference |
|---|---|---|---|
| Change from Baseline | | | (JZP-110 − |
| to the end of the | Placebo | JZP-110 | Placebo) |
| treatment period Parameter | (N = 34) | (N = 34) | (N = 34) |

Total Sleep Time (min)

| n | 30 | | 31 | | 31 | |
|---|---|---|---|---|---|---|
| Mean (SD) | 22.34 | (39.556) | −16.29 | (43.748) | −37.92 | (55.395) |
| Median | 16.19 | | −19.76 | | −34.31 | |
| Range | −53.3, 110.4 | | −86.4, 98.3 | | −150.1, 139.0 | |
| WASO (min) | | | | | | |
| n | 30 | | 31 | | 31 | |
| Mean (SD) | −2.55 | (9.721) | 1.45 | (14.701) | 3.91 | (12.943) |
| Median | −1.98 | | −0.69 | | 1.07 | |
| Range | −28.1, 14.6 | | −37.8, 50.5 | | −15.8, 52.1 | |
| Sleep Efficiency (%) | | | | | | |
| n | 30 | | 31 | | 31 | |
| Mean (SD) | 1.39 | (5.113) | −0.43 | (6.207) | −1.77 | (5.779) |
| Median | 1.94 | | −0.04 | | −1.83 | |
| Range | −9.4, 15.8 | | −20.5, 11.6 | | −14.8, 10.6 | |
| Sleep Onset Latency | | | | | | |
| for 24-hours (min) | | | | | | |
| n | 30 | | 31 | | 31 | |
| Mean (SD) | −7.18 | (21.091) | −6.44 | (21.698) | 0.50 | (22.215) |
| Median | −6.01 | | −4.75 | | −1.50 | |
| Range | −57.6, 33.1 | | −66.7, 36.7 | | −50.6, 76.1 | |
| Sleep Period (Time | | | | | | |
| in Bed) (min) | | | | | | |
| n | 30 | | 31 | | 31 | |
| Mean (SD) | 16.51 | (40.261) | −13.82 | (52.189) | −29.80 | (62.228) |

| | | | |
|---|---|---|---|
| Median | 13.08 | −23.37 | −28.00 |
| Range | −49.7, 110.4 | −81.2, 148.7 | −149.6, 177.7 |

min = minute;
mITT = modified intent-to-treat;
SD = Standard deviation;
N = number of subjects within each treatment assigned;
n = number of subjects with non-missing value;
WASO = Wake Time After Sleep Onset;

Total Sleep Time: Average total actual minutes of sleep obtained within a day (noon-noon). WASO: Average total awake time between initial sleep onset and final awakening from all sleep episodes within a day (noon-noon). Sleep Efficiency: Average sleep efficiency (Total Sleep Time/Time in Bed*100) from all sleep episodes within a day (noon-noon). Sleep Onset Latency for 24-hours: Average total latency between first attempting sleep and actual sleep onset from all sleep episodes within a day (noon-noon). Sleep Period (Time in Bed): Average time spent in bed/trying to sleep per day. Two subjects were not included in analysis due to incomplete data.

TABLE 8
Analysis: Psychomotor Vigilance Test (mITT Population)

| | | |
|---|---|---|
| Mean Reaction Time | Placebo | JZP-110 |
| PVT in msec | (N = 34) | (N = 34) |

2 Hours Post-dose

| | | |
|---|---|---|
| n | 33 | 34 |
| LS Mean (SE) | 318.66 (19.307) | 286.55 (19.004) |
| LS Mean Difference | | −32.111 |
| (JZP-110 − Placebo) | | |
| 95% CI | | −83.415, 19.193 |
| p-Value* | | 0.2144 |

6 Hours Post-dose

| | | |
|---|---|---|
| n | 33 | 33 |
| LS Mean (SE) | 341.37 (19.307) | 286.88 (19.188) |
| LS Mean Difference | | −54.494 |
| (JZP-110 − Placebo) | | |
| 95% CI | | −106.054, −2.934 |
| p-Value* | | 0.0387 |

ANOVA = analysis of variance;
N = number of subjects within each treatment assigned;
n = number of subjects with non-missing value;
PVT = Psychomotor Vigilance Test;
RT = reaction time

The PVT is administered at pre-dose and within 30 minutes before each driving test on Day 7 and 14. PVT is analyzed using a repeated mixed effect ANOVA model. The model includes treatment (JZP-110 and placebo), PVT test (2 hours post-dose and 6 hours post-dose), treatment period, treatment sequence and treatment by PVT test interaction as fixed effects and subject as a random effect. *p-values are nominal.

### Correlations Between Psychomotor Vigilance Test (PVT) and Driving Measures

Spearman correlations were used to explore the association between the driving measures (SDLP) and the PVT measures (lapses, mean RT, 1/RT). Results of these analyses for each of the different measures indicated correlations below an absolute value of 0.5 for both treatment groups (Table 9).

TABLE 9
Correlations between Standard Deviation of Lateral
Position and Psychomotor Vigilance Test

| | | | Placebo | JZP-110 |
|---|---|---|---|---|
| Time Point | PVT | Parameter | (N = 34) | (N = 34) |
| 2 Hours Post- | Number of Lapses (RT >500 msec) | r | 0.357 | 0.257 |
| dose | | | | |
| | Mean Reaction Time PVT in msec | r | 0.319 | 0.488 |
| | Inverse Reaction Time (1/RT[s]) | r | −0.315 | −0.457 |
| | Number of Errors of Commission | r | 0.200 | −0.264 |
| 6 Hours Post- | Number of Lapses (RT >500 msec) | r | 0.031 | 0.392 |
| dose | | | | |
| | Mean Reaction Time PVT in msec | r | 0.008 | 0.463 |
| | Inverse Reaction Time (1/RT[s]) | r | −0.130 | −0.445 |
| | Number of Errors of Commission | r | −0.289 | 0.108 |

N = number of subjects within each treatment assigned;
n = number of subjects with non-missing value;
r: spearman correlation coefficient;
RT = reaction time;
SDLP = standard deviation of lateral position

Driving Measures included SDLP at each time point. Number of errors of commission: number of responses without a stimulus, or false starts. Inverse reaction time: Each RT (ms) was divided by 1,000 and reciprocally transformed. The transformed values were then averaged.

### Sleep, Activity, Fatigue, and Task Effectiveness (SAFTE) Modeling

Biomathematical modeling was used to characterize changes in effectiveness associated with solriamfetol. Determining the impact of a novel medication on human performance in a sleep-disordered population requires comparing performance in the target population to controls with the same sleep history, but no sleep disorders or pharmaceutical interventions. Accomplishing this task in healthy-matched controls would be exceedingly difficult but could be achieved through biomathematical modeling.

Predicted performance in participants was assessed by inputting the time and date of each recorded sleep interval's start and end into the SAFTE model. The SAFTE model predicted cognitive performance based on the accumulation (or lack) of sleep over time, taking into account circadian processes and time of day. The model was well-validated in healthy populations, and predicted PVT data with exceptionally good accuracy, as well as performance degradation effects and the rate of recovery from schedules with restricted sleep (Hursh 2004). Biomathematical modeling of fatigue risk was a computational estimate of the effects of physiological fatigue on performance.

Performance in the SAFTE model is indicated as effectiveness, which was validated against speed of performance on the PVT based on parameters validated with a normal, shift-work population (Hursh, 2004). Effectiveness scores varied from 100 (typical best performance during the day) to zero. The PVT analyses were used to aid analyses in the SAFTE modeling.

### Psychomotor Vigilance Test

In this study, performance as measured by PVT (1/RT, mean RT, lapses) and calculated actual effectiveness were comparable following administration of solriamfetol compared to the predose time point or compared with performance after administration of placebo.

In this study, administration of solriamfetol was not related to any differences in PVT performance or sleep measures compared with placebo treatment for subjects with OSA, with the exception of mean RT at 6 hours postdose.

### Biomathematical Modeling and Changes in Effectiveness

Biomathematical (SAFTE) modeling of performance based on sleep history was predicted to be similar between placebo and solriamfetol conditions. Moreover, all measures of actual performance (1/RT, mean RT, lapses, errors of commission and calculated actual effectiveness) were also comparable following solriamfetol administration compared to the predose time point or placebo conditions.

Estimation of Risk Reduction Associated with Solriamfetol

Predicted effectiveness scores from SAFTE-FAST were not significantly different from actual effectiveness scores for either the placebo or solriamfetol for any PVT trial timepoints (all p>0.20).

Subjects' effectiveness was comparable to biomathematical performance predictions based on previous sleep which served as a proxy for healthy controls. Moreover, subjects' performance during either condition was similar to that of healthy sleepers under optimal sleep conditions measured under similar circumstances and time of day from an unrelated study on driving performance (Jongen 2015).

Biomathematical (SAFTE) modeling of performance based on sleep history indicated that all measures of actual PVT performance (1/RT, mean RT, lapses, errors of commission and calculated actual effectiveness) were comparable following solriamfetol administration compared to the predose time point or placebo conditions. Predicted effectiveness scores from SAFTE-FAST were also not different from actual effectiveness scores for either the placebo (t=−1.53, nominal p=0.13) or solriamfetol condition (t=0.14, nominal p=0.89) for any PVT trial time points (all nominal p>0.20).

### Safety Results

Treatment-emergent adverse events (TEAEs) were experienced by 17 (50.0%) subjects receiving solriamfetol compared with 11 (33.3%) subjects receiving placebo. There were no serious nor fatal TEAEs during this study. None of the subjects were withdrawn from the study nor from the study drug.

By preferred term (PT), events that occurred in 2 or more subjects (>5%; by decreasing frequency) included: headache, nausea, dizziness and insomnia. The majority of TEAEs were of mild severity (44.1% overall) or moderate severity (17.6% overall). Two subjects experienced severe TEAEs (placebo, headache; solriamfetol, nausea); the event of nausea was considered related to the study drug. A total of 16 subjects had at least 1 TEAE that was considered either related or suspected to be related to the study drug: 12 (35.3%) subjects who received solriamfetol and 7 (21.2%) subjects who received placebo. Study drug-related TEAEs that occurred in >5% subjects included, by decreasing frequency: headache, nausea, dizziness, insomnia and agitation

Three subjects reported suicidal ideation or suicidal behavior on the C-SSRS: 1 subject reported self-injury at the Screening Visit and 2 subjects reported behavior in response to death or illness in the family (1 subject at screening and 1 subject at Visit 5).

Mean changes from baseline in systolic and diastolic blood pressures (SBP and DBP, respectively) and pulse rate (PR) during the study were small and not dose-related and there were no clinically meaningful differences observed between treatment groups.

Mean values for clinical laboratory parameters were within normal ranges throughout the study. Mean and median changes were small, similar between solriamfetol and placebo groups, and were not clinically significant.

### Conclusion

In this randomized, double-blind, placebo-controlled, crossover, on-road driving study in subjects with excessive sleepiness due to OSA, solriamfetol demonstrated a statistically significant reduction in SDLP, at 2 hours postdose (the primary efficacy endpoint) and 6 hours postdose (secondary endpoint), as compared with placebo. Sensitivity analyses supported these findings. In the Psychomotor Vigilance Test, subjects who received solriamfetol prior to the test showed a trend towards fewer lapses, reduced reaction time, and a greater inverse reaction time as compared with those who received placebo. Similarly, subjects who received solriamfetol indicated a greater degree of alertness on the Toronto Hospital Alertness Test than those who received placebo.

Solriamfetol was well tolerated in this study. The most frequent adverse events (≥5%) were headache, nausea, dizziness and insomnia. There were no SAEs. One subject was withdrawn from the study due to sponsor decision; there were no withdrawals or discontinuations due to study drug. Sixteen subjects (47.1%) had a TEAE that was considered related to study treatment; most of these subjects (35.3%) experienced the event after receiving solriamfetol. The result for AEs, vital signs and laboratory tests displayed a safety profile similar to that observed in previous studies with solriamfetol, or was consistent with the underlying condition.

### Statistical Methods:

A sample size of 36 subjects provided 90% power to detect a mean difference of 2.0 cm on the primary outcome measure of SDLP. This calculation assumed a standard deviation of 3.25 cm and a 2-sided significance level of 0.05 using a paired t-test. To account for 10% dropouts without evaluable SDLP data, a sample size of approximately 40 subjects was planned.

All study data were summarized by treatment using descriptive statistics. Categorical results were reported as frequency and percent. Continuous variables were reported as number of subjects, mean, standard deviation, median, minimum, and maximum.

The primary outcome measure of mean change in SDLP was analyzed using a repeated mixed effect analysis of variance (ANOVA) model. The model included treatment (solriamfetol and placebo), driving performance tests (at 2 hours and 6 hours postdose), treatment period, and treatment by driving performance test interaction as fixed effects and subject as a random effect. The 2-sided 95% CIs of solriamfetol-placebo changes for SDLP based on the repeated mixed ANOVA model were constructed at each driving performance test. The assumption of normal distribution of the data required for ANOVA model was examined using the Shapiro-Wilk Normality test on the residuals from the mixed-effect model. The homogeneity of variance between treatments was evaluated using the Levene test. If the normality assumption and/or the homogeneity assumption was not satisfied at a significance level of 0.05, a non-parametric method (Wilcoxon signed-rank test) was used to compare the pair-wise treatment differences.

The secondary outcome measures of SDS, driving lapses (i.e., lane drift), THAT, and PVT measures were analyzed using a similar ANOVA method as used for SDLP.

The proportion of subjects with improved or impaired driving on solriamfetol compared with placebo was examined by maximally selected McNemar symmetry analyses. Spearman correlations were explored between driving measures (SDLP) and PVT measures (lapses, mean RT, 1/RT).

The incidence of treatment-emergent adverse events was summarized by treatment. Descriptive statistics were presented for vital sign results. No formal statistical testing was performed for the safety analyses.

### Measurements and Assessments

### Standardized Highway Driving Test

Assessments to determine driving performance included: standard deviation of lateral position (SDLP; Verster and Roth 2011; Raemakers 2017), standard deviation of speed (SDS), and number of driving lapses (also known as lane drift, defined as deviations>100 cm from the mean lateral position and from the absolute lateral position for 8 seconds; Vinckenbosch 2020). Driving performance was assessed using a standardized on-road driving test on Day 7 (Visit 4) and on Day 14 (Visit 5). A practice driving test was performed during the screening period to familiarize the subject with the vehicle and test scenario, assess if the subject could adequately operate the manual transmission vehicle, and determine if any safety concerns existed that excluded the subject from participating in the study.

During each drive, subjects operated a specially instrumented vehicle for approximately 1 hour over a 100 km (61 mile) primary highway circuit. During each driving test, each subject was accompanied by a licensed driving instructor who had access to dual controls (i.e., brakes, clutch, and accelerator). The subject or instructor could stop the test if either considered it unsafe to continue. The subject was instructed to drive with a steady lateral position between the delineated boundaries of the slower (right) traffic lane, while maintaining a constant speed of

95 km/h (58 mph). Subjects could deviate from these instructions only to pass a slower vehicle, to respond to a slower speed in traffic ahead of him/her and to leave and re-enter the highway at the turnaround point. During the drive, the vehicle's speed and lateral distance to the left lane line were continuously recorded and captured on an onboard computer disk file. Subjects were transported to and from the driving circuit on each test day.

### Psychomotor Vigilance Test

The PVT is a sustained-attention, reaction-timed task that measures the speed with which subjects respond to a visual stimulus. The PVT has been demonstrated to be sensitive to sleep disruption and is regarded as an objective indicator of cognitive impairment in a variety of conditions that result in sleepiness, including OSA (Dorrian 2005; Lim and Dinges 2008; Batool-Anwar 2014).

The PVT was administered at screening for practice only, and at predose and within 30 minutes before each driving test on Days 7 and 14 (Visits 4 and 5, respectively). The test was administered over 10 minutes with visual stimuli appearing randomly at variable intervals of 2 to 10 seconds. Subjects were instructed to respond to the appearance of a visual stimulus on a computer screen by pushing a response button as quickly as possible. The PVT measures included: mean RT, inverse reaction time (1/RT), lapses (RT>500 msec), and errors of commission.

### Toronto Hospital Alertness Test (THAT)

The THAT is a 10-item self-report questionnaire designed to measure perceived alertness in the preceding week (Shapiro 2006). The test was administered at baseline and prior to administration of study drug at Visits 4 and 5

### Actigraphy

Actigraphy is a method used to study sleep-wake patterns and circadian rhythms by assessing movement, most commonly of the wrist. Actigraph devices are generally placed on the wrist to record movement via detectors (e.g., accelerometers) and have sufficient memory to record for up to several weeks. Movement is sampled many times per second and stored for later analysis (Ancoli-Israel 2003). Computer programs were used to derive levels of activity/inactivity, rhythm parameters (such as amplitude or acrophase) and sleep/wake parameters (such as total sleep time, percent of time spent asleep, total wake time, percent of time spent awake, and number of awakenings). Actigraphy data can be used in combination with biomathematical modeling to estimate how an individual could be expected to perform in the absence of their central sleep disorder. The SAFTE biomathematical model of fatigue was designed and validated to predict the effects of fatigue on human performance (Hursh, Balkin et al, 2004; Hursh, Redmond et al, 2004). When used within its Fatigue Avoidance Scheduling Tool (FAST) application, the SAFTE-FAST tool can estimate an exposure to fatigue risk throughout the day.

Subjects wore an actigraph device on the wrist from Screening through Day 14 (Visit 5). The devices (“Actiwatch Spectrum” [sampling rate=32 Hertz], Philips Respironics; Bend, OR) were provided by the sponsor and dispensed at Screening. Subjects were reminded at both the clinical sites and at the driving test site regarding continued wear, and relevant instructions were reviewed with the subject, if needed, at each on-site visit or phone contact. Raw actigraphy data were scored by Philips Actiware software and using manual scoring techniques (Ancoli-Israel 2015) as described in the Institutes for Behavior Resources, Inc (IBR) SAFTE-FAST Modeling Report.

### Appropriateness of Measurements

The on-road driving test used for the study has been standardized and utilized in psychopharmacological research for over 30 years (Verster and Roth 2011; Raemakers 2017). The test conditions reflected actual driving and associated risks, and the safety of the driver was ensured by the presence of a licensed driving instructor who had access to dual controls. The primary outcome measure of vehicle control was the SDLP, which measures road-tracking error or amount of “weaving” of the vehicle. The SDLP is a sensitive outcome measure and driving impairment can be quantified to blood alcohol concentration (BAC) equivalent based on SDLP changes (Verster and Roth 2011; Raemakers 2017).

The PVT is a widely used and validated measure that is sensitive in the assessment of neurocognitive performance. The standard 10-minute PVT measures sustained or vigilant attention by recording RT to visual stimuli that occur at random inter-stimulus intervals. It offers a simple way to track changes in behavioral alertness caused by sleepiness without the confounding effects of aptitude and learning. It is highly reliable, within intra-class correlations for key metrics such as lapses measuring test-retest reliability above 0.8 (Dorrian 2005).

A combination of actigraphy and subject-reported daily sleep diary instead of polysomnography were used to conveniently record sleep/wake patterns continuously for 24-hours a day for the entire study duration. These methods were used in the study to ensure that subjects had adequate sleep and maintained a consistent sleep schedule in the study. The actigraphy, sleep diary, and PVT data were analyzed together with the driving measures in the development of a SAFTE model.

The use of vital signs, clinical laboratory tests, standard AE reporting, and the questionnaires that were selected to assess the safety of the study drug were appropriate since they are routinely used to assess the safety profile of drugs in clinical studies and pertinent to known risks of solriamfetol. The C-SSRS is able to determine clinically meaningful points at which a person may be at risk for an impending suicide attempt (Posner 2011).

### Analysis of Primary Endpoint

The primary analysis was based on the mITT population. The primary outcome measure of mean change in SDLP was analyzed using a repeated mixed effect analysis of variance (ANOVA) model. The model included treatment (solriamfetol and placebo), driving performance test (2 hours postdose and 6 hours postdose), treatment period, treatment sequence, and treatment by driving performance test interaction as fixed effects and subject as a random effect. The 2-sided 95% confidence intervals of solriamfetol-placebo changes for SDLP based on the repeated mixed effect ANOVA model was constructed for each driving performance test.

The assumption on normal distribution of the data required for ANOVA model was examined using the Shapiro-Wilk Normality test on the residuals from the mixed-effect model. The homogeneity of variance between treatments was evaluated using the Levene test. If the normality assumption and/or the homogeneity assumption were not satisfied at a significance level of 0.05, a non-parametric method (Wilcoxon Signed-Rank test) was used to compare the pair-wise treatment differences. A 5% type I error rate with a p-value<0.05 was considered statistically significant.

### Sensitivity Analysis of Primary Endpoint

The sensitivity analysis for the primary efficacy endpoint was conducted using the same statistical model as the primary analysis, but based on the PP population instead of the mITT population.

### Secondary Efficacy Endpoint Analyses

The secondary outcome measures of SDS, driving lapses, THAT, and PVT measures were analyzed using a similar ANOVA method as used for the primary endpoint. Spearman correlations were used to explore the association between driving measures (SDLP) and PVT measures (lapses, mean RT, 1/RT).

Individual changes (solriamfetol minus placebo) in driving performance were measured by SDLP at 2 hours and 6 hours postdose. The Maximum McNemar symmetry analyses (Laska et al. 2012) were used to detect an asymmetry in the distribution of the change in driving performance at 2 hours and 6 hours post-dose. The test examined the differences in the proportions of impaired drivers and improved drivers following treatment using a generalized sign test over all relevant thresholds. Individual improvement was defined as a decrease in SDLP below the negative value of threshold; individual impairment was defined as an increase in SDLP above the threshold or failure to complete the driving test due to sleepiness or subject-related safety concerns. If the maximum was larger than the critical value of the McNemar test for the pre-specified sample size and significance level, the difference in the proportions of subjects with improved or impaired driving performance was statistically significant. When a subject failed to complete the driving test under placebo or solriamfetol treatment, the following test status was applied for the analysis (Table 10).

In addition, single McNemar test statistics were obtained at each threshold (1.0, 1.5, 2.0, 2.5, 3.0, and 3.5 cm).

TABLE 10
Pre-specified McNemar Test Results Defined
by Driving Study Completion Status

| | |
|---|---|
| Treatment | Impaired or Improved |

| Placebo | JZP-110 | (JZP-110 minus Placebo) |
|---|---|---|
| Failed | Completed | Neither |
| Failed | Failed | Impaired |
| Completed | Failed | Impaired |
| Completed | Completed | Determined based on |
| | | defined threshold |

The Sleep, Activity, Fatigue and Task Effectiveness (SAFTE) modeling were generated by the IBR (Baltimore, MD), using data from PVT, actigraphy, and the sleep diary.

Psychomotor Vigilance Test performance was used to calibrate the model to the effects of solriamfetol. Thereafter, biomathematical modeling was used to estimate the anticipated reduction in risk that would be associated with solriamfetol. Spearman correlations were explored between driving measures (SDLP) and each of the PVT measures (lapses, mean RT, 1/RT).

Subjects' average 1/RT per PVT trial was used to calculate an actual effectiveness score, which was then compared to SAFTE-FAST predicted effectiveness. SAFTE-FAST effectiveness predictions have been validated against 1/RT, or the speed of performance, on a PVT (Hursh 2004). Actual effectiveness between drug conditions was then compared to predicted effectiveness using paired samples t-test, controlling for drug condition and test session. Repeated measures ANOVA was additionally performed to compare the effect of treatment conditions (solriamfetol versus placebo) on actual effectiveness over time. Sleep data were scored and the sleep, activity, and daily summary intervals were exported from the Actiware. The variables from actigraphy (total sleep time, wake time after sleep onset [WASO], sleep period, sleep efficiency, sleep onset latency for 24-hours, and night sleep periods), derived from each treatment period and the change from baseline, were summarized. The baseline value was derived from the average of measurements from 7 days prior before the first dose. The postbaseline value was the average of measurements collected in the treatment period.

Sleep intervals were used for SAFTE-FAST modeling. Some sleep periods were missing due to missing data (off-wrist or watch malfunction) or misidentification of sleep periods by the algorithm. For modeling purposes, if sleep diary information was available for the interval of missing data, that sleep interval was added into the schedule. All statistical analyses were completed using STATA 15.1 and Excel 2016.

### Actigraphy

The following variables from actigraphy were derived from each treatment period and the change from baseline was summarized:
Total sleep time: Average total actual minutes of sleep obtained within a day (noon-noon) Wake time after sleep onset (WASO): Average total awake time between initial sleep onset and final awakening from all sleep episodes within a day (noon-noon)
Sleep period (time in bed): Average time spent in bed/trying to sleep per day Sleep efficiency: Average sleep efficiency (Total Sleep Time/Time in Bed*100) from all sleep episodes within a day (nooWASn-noon)
Sleep onset latency for 24-hours: Average total latency between first attempting sleep and actual sleep onset from all sleep episodes within a day (noon-noon)
Night sleep periods: The baseline values were derived using the average of all measurements taken prior to the first dose. This was consistent with the SAFTE modeling conducted by IBR. The post-baseline value was the average of measurements collected in the treatment period.

### Safety

This short-term, randomized study of subjects with OSA showed solriamfetol was tolerated at doses of 150 and 300 mg QD over 7 days. No SAEs occurred during the study. The most frequent AEs (>5%) were headache, nausea, dizziness and insomnia. Most of these events were mild to moderate in severity; there were 2 severe events (placebo, headache; solriamfetol, nausea). One subject was withdrawn from the study prior to receiving placebo due to sponsor decision. The results for AEs, vital signs, and laboratory tests displayed a safety profile similar to that observed in previous studies with solriamfetol.

### Conclusions

In this randomized, double-blind, placebo-controlled, crossover, on-road driving study in subjects with excessive sleepiness due to OSA, solriamfetol demonstrated a statistically significant reduction in SDLP at 2 hours postdose (the primary efficacy endpoint) and 6 hours postdose (the secondary endpoint) as compared with placebo. Sensitivity analyses supported these findings. In the PVT, subjects who received solriamfetol prior to the test showed a trend towards fewer lapses, reduced reaction time, and a greater inverse reaction time as compared with those who received placebo. Similarly, subjects who received solriamfetol indicated a greater degree of alertness on the THAT than those who received placebo.

Solriamfetol was well tolerated in this study. The most frequent AEs (>5%) were headache, nausea, dizziness and insomnia. There were no SAEs. One subject was withdrawn from the study due to sponsor decision; there were no withdrawals or discontinuations due to study drug.

Sixteen subjects (47.1%) had a TEAE that was considered related to study drug; most of these subjects (35.3%) experienced the event after receiving solriamfetol. The result for AEs, vital signs, and laboratory tests displayed a safety profile similar to that observed in previous studies with solriamfetol, or was consistent with the underlying condition.

### REFERENCES

American Academy of Sleep Medicine (AASM). International Classification of Sleep Disorders-Third Edition (ICSD-3), Darien, I L. American Academy of Sleep Medicine. 2014; 53-62, 143-55.
Ancoli-Israel S, Cole R, Alessi C, et al. The role of actigraphy in the study of sleep and circadian rhythms. Sleep. 2003 May 1; 26(3):342-92.
Ancoli-Israel S, Martin J L, Blackwell T, et al. The SBSM guide to actigraphy monitoring: clinical and research applications. Behav Sleep Med. 2015; 13 Suppl 1:S4-S38.
Batool-Anwar S, Kales S N, Patel S R, Varvarigou V, DeYoung P N, Malhotra A. Obstructive sleep apnea and psychomotor vigilance task performance. Nat Sci Sleep. 2014; 6:65-71.
Barbe F, Pericas J, Munoz A, et al. Automobile accidents in patients with sleep apnea syndrome. An epidemiological and mechanistic study. Am J Respir Crit Care Med 1998; 158(1): 18-22.
Broderick J E, Junghaenel D U, Schneider S, Pilosi J J, Stone A A. Pittsburgh and Epworth sleep scale items: accuracy of ratings across different reporting periods. Behav Sleep Med. 2013; 11:173-88.
Dinges D F, Pack F, Williams K, et al. Cumulative sleepiness, mood disturbance, and psychomotor vigilance performance decrements during a week of sleep restricted to 4-5 hours per night. Sleep 1997; 20(4): 267-277.
Dorrian J, Rogers N L, Dinges D F, Kushida C A. Psychomotor vigilance performance: Neurocognitive assay sensitive to sleep loss. Sleep deprivation: clinical issues, pharmacology and sleep loss effects. New York, N Y: Marcel Dekker, Inc.; 2005. pp 39-70.
Durmer J S, Dinges D F. Neurocognitive consequences of sleep deprivation. Semin Neurol 2005; 25(1): 117-129.
George C F. Sleep apnea, alertness, and motor vehicle crashes. Am J Respir Crit Care Med. 2007; 176:954-6.
Hayley A C, Williams L J, Kennedy G A, et al. Prevalence of excessive daytime sleepiness in a sample of the Australian adult population. Sleep Med. 2014; 15(3):348-54.
Hursh S R, Redmond D P, Johnson M L, et al. Fatigue models for applied research in warfighting. Aviat Space Environ Med 2004; 75(3 Suppl): A44-60
Jaussent I, Morin C M, Ivers H, Dauvilliers Y. Incidence, worsening and risk factors of daytime sleepiness in a population-based 5-year longitudinal study. Sci Rep. 2017; 7(1):1372.
Johns, M W. Sensitivity and specificity of the multiple sleep latency test (MSLT), the maintenance of wakefulness test and the Epworth Sleepiness Scale: failure of the MSLT as a gold standard J Sleep Res 2000; 9 (1): 5-11.
Johns, M W. A new method for measuring daytime sleepiness: the Epworth sleepiness scale. Sleep. 1991; 14(6):540-5.
Jongen S, Perrier J, Vuurman E F, et al. Sensitivity and validity of psychometric tests for assessing driving impairment: effects of sleep deprivation. PLoS One 2015; 10(2).
Jongen S, Vermeeren A, Van Der Sliszen Nnjjm, et al. Phychopharm. 2017; 234(5): 837-44.
Joo S, Baik I, Yi H, et al. Prevalence of excessive daytime sleepiness and associated factors in the adult population of Korea. Sleep Med. 2009; 10(2):182-8.
Kay G G, Feldman N. Effects of armodafinil on simulated driving and self-report measures in obstructive sleep apnea patients prior to treatment with continuous positive airway pressure. J Clin Sleep Med. 2013; 15: 445-54.
Laska E, Meisner M, Wanderling J. A maximally selected test of symmetry about zero. Stat. Med. 2012:31(26):3178-91.
Lee I S, Bardwell W A, Ancoli-Israel S, et al. Number of lapses during the psychomotor vigilance task as an objective measure of fatigue. J Clin Sleep Med 2010; 6(2): 163-168.
Li Y, Vgontzas A, Kritikou I, et al. Psychomotor vigilance test and its association with daytime sleepiness and inflammation in sleep apnea: clinical implications. J Clin Sleep Med 2017; 13(9): 1049-1056.
Lim J, Dinges D F. Sleep deprivation and vigilant attention. Ann NY Acad Sci. 2008; 1129:305-22.
Posner K, Brown G K, Stanley B, et al. The Columbia-Suicide Severity Rating Scale: initial validity and internal consistency findings from three multisite studies with adolescents and adults. Am J Psychiatry. 2011; 168:1266-77.
Ramaekers J G, Kuypers K P, Samyn N. Stimulant effects of 3,4-methylenedioxymethamphetamine (MDMA) 75 mg and methylphenidate 20 mg on actual driving during intoxication and withdrawal. Addiction. 2006; 101:1614-21.
Ramaekers J G, Kuypers K P, Bosker W M, et al. Effects of stimulant drugs on actual and simulated driving: perspectives from four experimental studies conducted as part of the DRUID research consortium. Psychopharmacology (Berl). 2012; 222 (3):413-8.
Ramaekers J G. Drugs and driving research in medicinal drug development. Trends Pharmacol Sci. 2017; 38(4):319-21.
Shapiro C M, Auch C, M. Kayumov R L, Heslegrave R; Huterer N, Driver H, Devins C M. A new approach to the construct of alertness. Journal of Psychosomatic Research. 2006; 60 (6):595-603.
Tregear S, Reston J, Schoelles K and Phillips B. continuous positive airway pressure reduces risk of motor vehicle crash among drivers with obstructive sleep apnea: systematic review and meta-analysis. Sleep. 2010; 33: 1373-80.
Verster J C, Bekker E M, de Roos M. Methylphenidate significantly improves driving performance of adults with attention-deficit hyperactivity disorder: a randomized crossover trial. J Clin Psychopharmacol. 2008; 22:230-37.
Verster J C, Roth T. Standard operation procedures for conducting the on-the-road driving test, and measurement of the standard deviation of lateral position (SDLP). Int J Gen Med. 2011; 4:359-71.
Vinckenbosch F R J, Vermeeren A, Verster J C, et al. Validating lane drifts as a predictive measure of drug or sleepiness induced driving impairment. Psychopharmacol (Berl). 2020; 237:877-886. Wu S, Wang R, Ma X, et al. Excessive daytime sleepiness assessed by the Epworth sleepiness scale and its association with health related quality of life: a population-based study in China. BMC Public Health. 2012; 12:849.

### Example 3. Psychomotor Vigilance Performance in Participants with Excessive Daytime Sleepiness in Obstructive Sleep Apnea or Narcolepsy Compared with SAFTE-FAST Model Predictions

### Introduction

In clinical settings, attention and vigilance can be examined using a variety of measures, including the psychomotor vigilance task (PVT), a measure of sustained attention that correlates with subjective sleepiness [14]. Furthermore, sleep-related declines in PVT function correlate with poor performance on driving measures, such as lane drift [15]. Compared with healthy controls, patients with sleep disorders and sleep deprivation perform poorly on the PVT [16,17]. However, some commonly used PVT metrics have been found to lack sensitivity, and differential vulnerability to poor performance on the PVT has been noted in a subset of healthy individuals [18,19]. Additionally, measures commonly used to assess performance in clinical trials rarely overlap with those used in occupational settings.

When evaluating treatments for EDS secondary to sleep disorders, comparisons can be made between patients and healthy controls, and/or between patients receiving the treatment of interest and those receiving placebo, to determine the impact on performance in the target population. Ideally, comparisons would be made against a control group with the same sleep history; however, current approaches do not control for differences in sleep behavior (as either a function of disease history or drug effect) between individuals. Because differences in sleep behavior can affect outcomes of interest, such as measures of attention [20], the ability to isolate the specific effects of treatments on such outcomes can be confounded. Integrating biomathematical modeling, as used in occupational assessments of performance, may help compensate for shortcomings related to control of sleep behavior in clinical research.

The Sleep, Activity, Fatigue, and Task Effectiveness (SAFTE) biomathematical model has been used to predict the effects of fatigue on performance [21] in healthy individuals [22]. This model predicts cognitive performance based on several input parameters, including the accumulation of sleep over time, taking into account circadian processes and time of day. Pairing the SAFTE model with the Fatigue Avoidance Scheduling Tool (FAST) has provided a means (SAFTE-FAST) to estimate and mitigate exposure to fatigue risk throughout the day. The SAFTE model has widespread use among transportation (e.g., aviation, rail) agencies and the US Department of Defense, where it allows for the construction of schedules that avoid performance impairment attributable to fatigue [22]. The SAFTE model has previously been demonstrated to predict human factors-related freight rail accident risk based on predicted fatigue-induced impairments [23]. Analysis showed that the relative risk was increased 42% when SAFTE-predicted effectiveness scores were <77%, but was reduced by 30% when scores were >90%. However, the SAFTE model has not been investigated in patients with sleep disorders. In the novel application described herein, the SAFTE model functioned as a proxy for healthy controls by predicting the treatment- and time-dependent effects that would have been expected had the participants been healthy controls with exactly the same sleep history as the participants from whom data were collected.

As described in Examples 1 and 2, the effect of solriamfetol on real-world driving was recently examined in two randomized, crossover, placebo-controlled phase 2 trials in participants with narcolepsy or OSA [33,34]. In both studies, solriamfetol significantly decreased (improved) standard deviation of lateral position (SDLP) at 2 hours post dose, meeting the primary efficacy endpoints. PVT and actigraphy data were also collected during these trials.

The aim of this analysis was to explore the utility of the SAFTE model as a substitute for a healthy control group using predicted PVT performance based on actigraphy-derived sleep parameters compared with actual PVT performance from these two solriamfetol phase 2 clinical trials.

Evaluating the effects of treatments for excessive daytime sleepiness, such as solriamfetol, on task performance is challenging because of differences in sleep history and sleep behavior between individuals. These analyses explored the use of the Sleep, Activity, Fatigue, and Task Effectiveness (SAFTE) biomathematical model as a proxy for healthy controls using psychomotor vigilance task data from two placebo-controlled clinical trials of solriamfetol.

Data were analyzed from two phase 2 studies of solriamfetol in adults with OSA (NCT02806895, EudraCT 2015-003930-28) or narcolepsy (NCT02806908, EudraCT 2015-003931-36). Participants were randomly assigned 1:1 to solriamfetol 150 mg/day (3 days) followed by 300 mg/day (4 days), or placebo (7 days), then crossed over to the other treatment. Actual task effectiveness scores were calculated from average PVT inverse reaction time (pre-dose, 2 hours post-dose, 6 hours post-dose). Actigraphy-derived sleep intervals were used in SAFTE to determine modeled healthy control task effectiveness scores.

In participants with OSA (N=31) on placebo or solriamfetol, actual and modeled healthy control task effectiveness did not differ at any timepoint. In participants with narcolepsy (N=20) on placebo, actual task effectiveness at 2 hours post-dose was lower than modeled healthy control task effectiveness (nominal P=0.03), a difference not present with solriamfetol. There was no main effect of solriamfetol on actual or modeled healthy control task effectiveness across timepoints.

### Results

This report summarizes the effect of solriamfetol treatment on driving performance in subjects with OSA. In this double-blind, crossover, on-road driving study, subjects received either solriamfetol (150 mg QD for 3 days, followed by 300 mg QD for 4 days) or matching placebo for 7 days, and then crossed over to receive the other treatment regimen for 7 days. On Day 7 of each treatment period, subjects undertook driving performance tests, at 2 hours and at 6 hours postdose, along with other efficacy assessments testing for alertness and response time.

For the primary endpoint, SDLP at 2 hours postdose, solriamfetol demonstrated a statistically significant improvement compared with placebo (p=0.0062). Overall, the mean SDLP at 2 hours was less in subjects who received solriamfetol as compared with placebo: 18.83 cm versus 19.92 cm, respectively, an LS mean difference −1.084 cm. Similarly, there was a statistically significant improvement compared with placebo at 6 hours postdose: −0.804 cm (p=0.0432).

Sustained attention is a necessary requirement for the safe operation of a motor vehicle in traffic (Vinckenbosch 2020). It has been estimated that up to 15% of the traffic accidents on motor ways are associated with sleepiness (Maycock, 1996). The SDLP is the main outcome measure of the on-road highway driving test, which is generally regarded as a gold standard for assessing drug-induced driving impairment (Jongen 2017). Subjects driving with a blood alcohol concentration of 0.5 g/L (0.05%) show mean SDLP increases of approximately 2.5 cm relative to those driving under placebo, and epidemiological studies have demonstrated that crash risk increases at concentrations exceeding this threshold (Borkenstein et al. 1974).

Driving studies have demonstrated that sleep deprivation or the use of sedative medications (e.g., benzodiazepines, antidepressants) can also produce increments in SDLP equal to or greater than the 2.5 cm clinical threshold (Jongen 2018; Veldhuijzen 2006; Jongen 2015). In this study, subjects with OSA showed a mean difference of approximately −1.084 cm in SDLP when driving after receiving placebo as compared to solriamfetol, indicating greater deviation on placebo.

While not reaching the threshold of 2.5 cm, these results underscore the degree of impairment that subjects with OSA may experience when driving. The PVT is a validated test of attention that shows sensitivity to sleep loss and is independent of aptitude (Dinges 1997; Durmer and Dinges 2005; Lim and Dinges 2008). While the PVT has emerged as one of the most widely used tools to assess vigilant attention in sleep research (Dinges 1997), it is not widely used to assess vigilance in sleep disorders like OSA. In previous studies, PVT performance has been shown to be related to subjective reports of daytime sleepiness, but not physiological measures of sleep in OSA (Barbe 1998; Lee 2010; Batool-Anwar 2014; Li 2017).

Performance in the SAFTE model, indicated as effectiveness, has been validated against speed of performance on the PVT based on parameters validated with a normal, shift-work population (Hursh 2004). In this study, sleep history and SAFTE-predicted performance were compared between solriamfetol and placebo control conditions and against PVT data collected from subjects with OSA during the clinical study. In the current study, the SAFTE-FAST tool was used to model performance based on objective actigraphy sleep collected from an OSA population to evaluate the effects of solriamfetol on performance, independent of sleep behavior in subjects with OSA.

### Demographics

For participants with OSA (safety population, n=34; detailed demographics and disposition previously reported) [33,34], 88% were male, mean (SD) age was 51.6 (12.3) years, mean (SD) MWT sleep latency was 14.3 (7.3) minutes, and 85% used a primary OSA therapy.

For participants with narcolepsy (safety population, n=24; detailed demographics and disposition previously reported) [33,34], 54% were male, mean (SD) age was 40.4 (11.8) years, and mean (SD) MWT sleep latency was 4.0 (2.5) minutes (MWT latencies were based on historical data and were not collected as part of the study; n=22).

Participants with missing sleep data (due to noncompliance or device errors) were excluded from analyses of PVT data and biomathematical modeling. Data were analyzed for 31 participants with OSA and 20 participants with narcolepsy.

### Use of SAFTE Biomathematical Modeling to Characterize Task Effectiveness Changes

Participants with OSA

In participants with OSA, analysis of actual task effectiveness showed no main effect of treatment or treatment×time interaction effects based on repeated-measures ANOVA (Table 11); however, there was an effect of previous day's sleep (P=0.001). In a regression analysis, longer sleep the night before testing was not independently related to actual effectiveness (R2=0.01, P=0.98), suggesting this relationship was nonlinear. Actual task effectiveness was lower in participants treated with solriamfetol compared with placebo at pre-dose (P=0.02) but not at 2 or 6 hours post-dose (P>0.05 for both).

In participants with OSA, repeated-measures ANOVA of modeled healthy control task effectiveness showed no main effect of treatment or treatment×time interaction effects; however, similarly to actual task effectiveness, there was an effect of previous day's sleep (P=0.04). Longer sleep was independently related to higher modeled healthy control task effectiveness (R2=0.47, P<0.001).

There were no differences between modeled healthy control and actual task effectiveness scores for solriamfetol or for placebo in participants with OSA (P>0.05 overall and for all timepoints) (FIG. 6).

Participants with Narcolepsy

In participants with narcolepsy, repeated-measures ANOVA showed a treatment×time interaction effect (P<0.001) on actual task effectiveness, with task effectiveness improving after administration of solriamfetol (from pre-dose to 2 hours post-dose to 6 hours post-dose) but deteriorating after administration of placebo (Table 11). Additionally, there was a main effect of previous day's sleep (P=0.004), similar to that observed in participants with OSA. Longer sleep the night before testing was independently related to poorer actual effectiveness (R2=0.21, P<0.001)

There was no main effect of treatment or treatment×time interaction effects on modeled healthy control task effectiveness based on repeated-measures ANOVA; however, in line with other analyses, there was an effect of previous day's sleep on modeled healthy control task effectiveness (P<0.001). Longer sleep was independently related to higher modeled healthy control task effectiveness (R2=0.47, P<0.001).

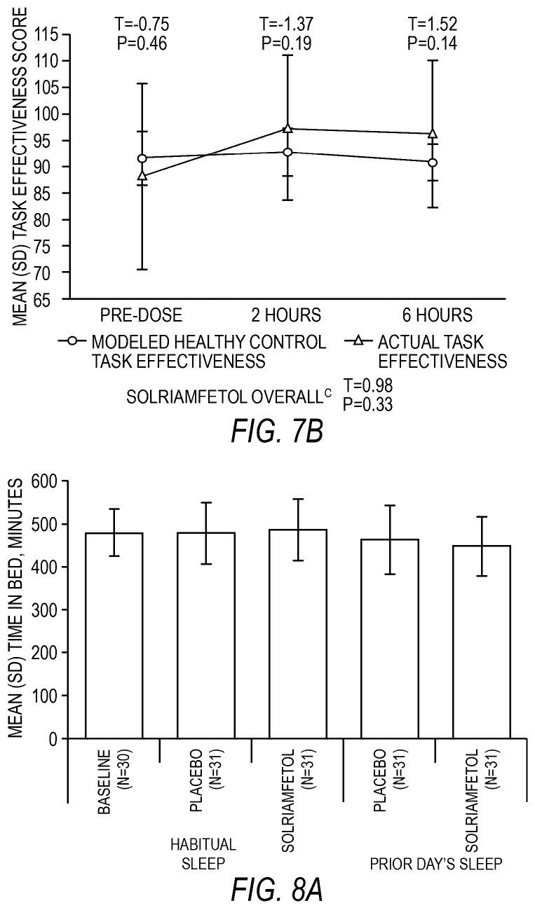

Modeled healthy control task effectiveness was higher than actual task effectiveness for placebo (P=0.009), primarily driven by PVT performance at 2 hours post-dose (P=0.03) in participants with narcolepsy. Modeled healthy control and actual task effectiveness did not differ for solriamfetol (P>0.05 overall and at all timepoints) (FIG. 7).

### Change in SAFTE Model Input Parameters and PVT Measures

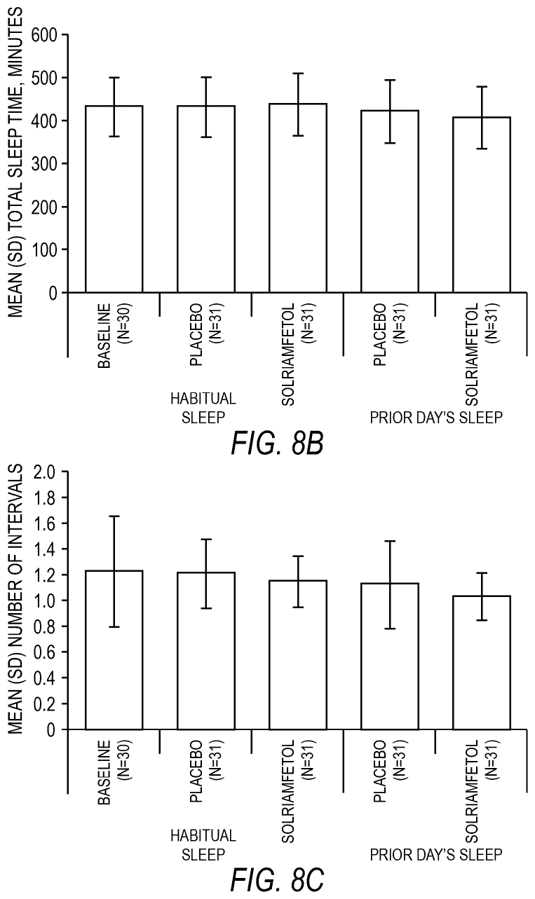

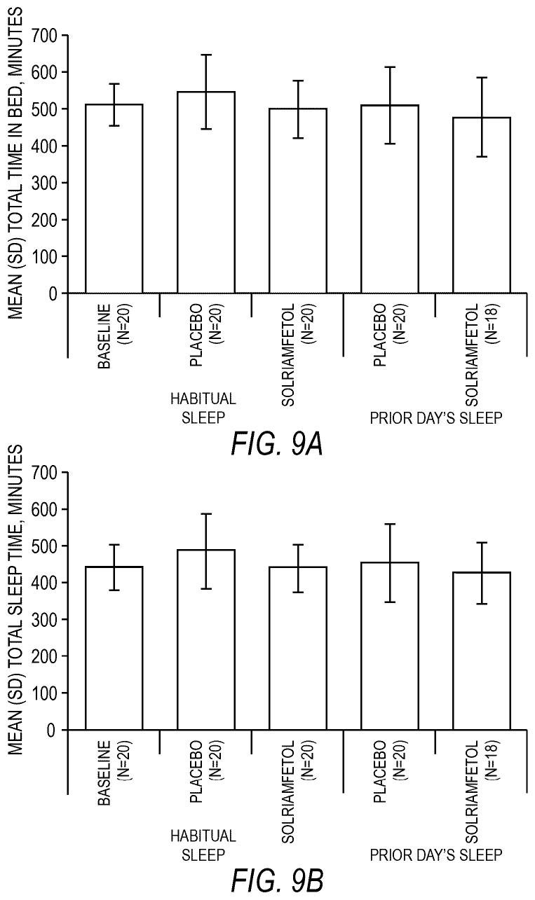

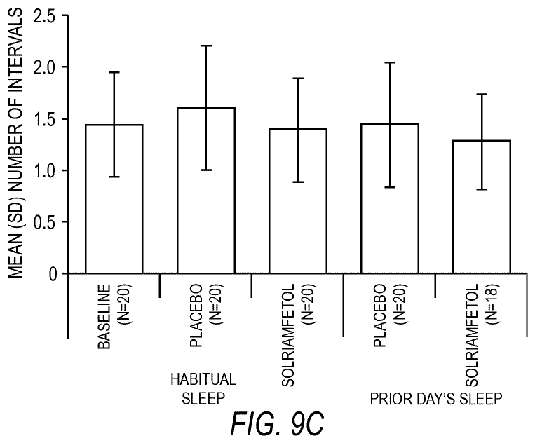

In participants with OSA or narcolepsy (FIG. 8 and FIG. 9, respectively), there were no differences between placebo and solriamfetol treatment in either habitual sleep measures (TIB, TST, DSIs) or TIB, TST, or DSIs measured the day before PVT testing.

There was no main effect of solriamfetol on any PVT measure in participants with OSA (Table 12; repeated-measures ANOVA, P>0.05 for all) or participants with narcolepsy (Table 13; repeated-measures ANOVA, P>0.05 for all).

### Discussion

In this analysis, SAFTE modeling data (i.e., modeled healthy control task effectiveness) were used as a proxy for healthy participants in a clinical trial. This represents a novel application of SAFTE, which previously has been used to model the effects of sleep deprivation in healthy populations [21,22]. In participants with OSA, actual task effectiveness did not differ from modeled healthy control task effectiveness with placebo or with solriamfetol, despite participants' EDS (evidenced by a MWT mean sleep latency of 14 minutes at baseline). In light of this finding (i.e., an apparent lack of a deficit in performance in participants with OSA), it is unsurprising that actual task effectiveness did not differ between placebo and solriamfetol in participants with OSA. In participants with narcolepsy, actual task effectiveness with placebo was lower than modeled healthy control task effectiveness (suggesting a deficit in performance in participants with narcolepsy), whereas actual task effectiveness with solriamfetol did not differ from modeled healthy control effectiveness; actual task effectiveness improved with solriamfetol relative to placebo. Thus, in contrast to the differences in SDLP between solriamfetol and placebo (reported elsewhere), the present findings suggest that, in these participants with OSA, task effectiveness while on placebo or solriamfetol was similar to that of “healthy controls,” despite the participants' sleep disorder. In participants with narcolepsy, however, task effectiveness while on placebo was lower than that of “healthy controls” and improved on solriamfetol to a level similar to “healthy controls.”

Analyses of the primary outcomes of these studies demonstrated that solriamfetol improved real-world, on-road driving performance as measured by SDLP at 2 hours post-dose in participants with narcolepsy as well as participants with OSA. Therefore, the SAFTE model findings can be considered generally consistent with the primary study findings for participants with narcolepsy. In contrast, the results for participants with OSA suggest that, despite their degree of EDS, they had a relative lack of impairment in PVT performance and task effectiveness scores while on placebo. Although the small sample sizes preclude any definitive conclusions, it is possible that the lack of detectable improvement may be due to a “ceiling effect.” This finding may indicate that PVT performance is not sensitive enough to detect impairment attributable to EDS in OSA in a study of this size.

There was no effect of solriamfetol treatment on any of the sleep measures recorded in participants with OSA or participants with narcolepsy. This finding aligns with data from phase 3 trials of solriamfetol in participants with narcolepsy and OSA in which sleep measured by polysomnography was unchanged with solriamfetol compared with placebo [25,26]. However, as sleep is a fundamental input for the SAFTE model, the lack of an effect of solriamfetol on sleep measures is a key consideration in interpreting the present findings.

This analysis found no effect of solriamfetol on PVT metrics in participants with OSA; however, participants with narcolepsy were faster (IRT) and had fewer lapses after treatment with solriamfetol compared with placebo. These results are consistent with the SAFTE findings. However, previous studies established the efficacy of solriamfetol in reducing EDS in patients with OSA or narcolepsy [26,28,30], and the primary results of the present studies demonstrated that solriamfetol improved real-world, on-road driving performance as measured by SDLP at 2-hours post-dose in both groups of participants. As this study was powered for SDLP rather than PVT or SAFTE, it may be that larger studies are needed to observe further differences in participants with OSA.

Several factors should be kept in mind when considering the PVT findings in particular. Most important, these studies had small sample sizes and were not powered to detect a difference in PVT scores with solriamfetol treatment. In patients with OSA, PVT scores were previously found not to differ by apnea-hypopnea index severity [41], and have been shown to be associated with subjective (ESS) but not objective (MWT) sleepiness in patients with OSA [14]. Further, PVT performance in patients with narcolepsy has been found to be worse than in patients with insufficient sleep syndrome [16].

The real-world driving performance as measured by SDLP (an established correlate of drug- and alcohol-induced motor vehicle crash risk [42]) was improved at 2 hours post-dose in both studies [33,34]. SAFTE-modeled healthy control task effectiveness was not examined in the context of real-world driving performance in healthy controls in this study or in previous studies. The SAFTE model was not designed as a performance predictor for SDLP. If in future studies investigators intend to use a biomathematical model in lieu of a healthy control population, power analyses should be performed to reflect PVT performance or similar model-tested performance outcomes a priori. The SAFTE model has not been optimized for the prediction of performance or risk in people with sleep disorders.

While the goal for the current analysis was to use SAFTE-FAST as a proxy for healthy controls, some lessons can be extrapolated to the application of biomathematical modeling to sleep disorders. For this analysis, the model used patient TIB and assumed excellent sleep quality, as the goal of the model was to serve as a proxy for healthy controls. Patients with sleep disorders often have sleep fragmentation (e.g., because of apneas, as in patients with OSA), which may not be apparent in actigraphy data and is not considered when computing TIB. SAFTE-FAST can use TST instead of TIB for a sleep input, and users can modify the sleep quality setting to reflect disrupted sleep. Using TST or adjusting the sleep quality of sleep events could help replicate the objective fragmentation of sleep seen in sleep disorders. One area of limitation is that the model assumes a normal relationship between sleep duration and changes in performance, but this relationship may be weaker in patients with narcolepsy [43]. The weakening of this relationship may explain why longer prior day's sleep was not independently correlated with improved actual task effectiveness in either group. The biological underpinnings that could explain the breakdown between sleep and performance in narcolepsy need to be better understood before a biomathematical model can reasonably be developed.

This novel application of the SAFTE model in a clinical trial setting highlights the need for additional metrics and thereby informs future studies. The crossover design used here strengthens the validity of its comparisons by controlling for interindividual differences between treatment conditions. Indeed, although they provide useful safety benchmarks, neither PVT nor SAFTE-FAST task effectiveness scores are used in isolation as determinants of ability to drive or perform other tasks. Aviation and other industries want to limit the amount of time spent below 77% effectiveness, which is equivalent to a 0.05% blood alcohol concentration [44]. In the present analysis of participants with OSA or narcolepsy, both populations had actual task effectiveness levels above this cutoff based on the model as implemented; however, these findings should not be over-interpreted as an equivocal statement of safety, especially given the extensive published literature demonstrating the increased risk of motor vehicle crashes in these populations [9,10,45-48].

### Conclusions

This study represents a novel application of the SAFTE-FAST biomathematical model as a substitution for healthy controls in a research investigation of patients with sleep disorders. The results provide valuable lessons that may help to optimize future studies that incorporate this model to ensure the applicability of their results. For example, future studies should examine the use of sleep measures (other than TIB) that reflect the objective fragmentation of sleep seen in sleep disorders. This analysis also provides additional context for how PVT performance during a clinical trial may differ from performance on real-world performance measures, such as lane drift.

### Methods

### Study Design

This analysis used data collected during two phase 2 studies examining the effects of solriamfetol on driving performance in participants with OSA (NCT02806895, EudraCT 2015-003930-28) or narcolepsy (NCT02806908, EudraCT 2015-003931-36) [33,34]. The SAFTE modeling analyses were planned secondary efficacy endpoints for both trials. In these trials, participants were randomly assigned 1:1 to receive solriamfetol 150 mg/day for 3 days followed by 300 mg/day for 4 days, or placebo for all 7 days (Period 1), and then were crossed over to the other 7-day treatment (Period 2). As the studies were conducted before finalization of regulatory approval or dosing recommendations, the dose of 300 mg/day was based on prior phase 2 clinical trial results [35,36] and aligned with the maximum dose used in the phase 3 clinical trials [26-28,30].

The study protocols were approved by the medical ethics committee of University Hospital Maastricht and Maastricht University (www.toetsingonline.nl; OSA, NL56214.068.16; narcolepsy, NL56215.068.16), and all participants provided written informed consent. The primary endpoint for both studies was the effect of solriamfetol on on-road driving performance, as assessed by SDLP at 2 hours post-dose (reported separately).

### Participants

As reported previously [33,34], participants ranged in age from 21 to 75 years, had either an OSA diagnosis per International Classification of Sleep Disorders—Third Edition (ICSD-3) or a narcolepsy diagnosis per ICSD-3 or Diagnostic and Statistical Manual of Mental Disorders, Fifth Edition, and an average total nightly sleep≥6 hours. Additional inclusion criteria for participants with OSA included baseline Epworth Sleepiness Scale (ESS) score≥10, mean sleep latency<30 minutes on the Maintenance of Wakefulness Test (MWT), and 1 of the following: use of a primary therapy for OSA≥1 night per week with ≤2 days of variation week to week, lack of use of primary therapy after a history of ≥1 month of attempted use with ≥1 documented adjustment, or a history of surgical intervention. Participants who were unwilling to attempt to use 1 or more primary OSA therapies were excluded.

### Procedures

Participants were instructed to take a single capsule containing their treatment once daily, within 1 hour of waking in the morning, on an empty stomach, and then to wait≥30 minutes before having breakfast. At visits on Days 7 and 14, participants completed the PVT along with an on-road driving test and other measures (reported separately) [33,34]. On test days, the PVT was administered pre-dose and at approximately 2 hours and 6 hours post-dose. The PVT was administered over 10 minutes, with visual stimuli appearing at random variable intervals of 2 to 10 seconds; participants responded to a digital signal by pressing a key on a computer terminal. Participants also wore an actigraph from screening through Day 14 (except during testing sessions, as data were extracted during that time) and kept accompanying sleep diaries.

### Assessments and Outcomes

Participants completed the PVT by responding to a digital signal on a computer terminal via key presses, as described above. Calculated PVT measures included mean reaction time (MRT), lapses (reaction times>500 ms or failures to respond), and errors (responses made without stimulus or false starts).

Sleep metrics, including time in bed (TIB), total sleep time (TST), and daily sleep intervals (DSIs), were measured at baseline and throughout the treatment period. Raw actigraphy data were scored using Actiware software (Philips Respironics, Bend, OR) and manual scoring techniques [37]. Sleep diaries were used to assess the major sleep interval start and stop times and any naps not scored by the Philips algorithm but confirmed by activity pattern. For manual scoring, sleep diaries were compared with actigraphy data.

### Task Effectiveness

Actual task effectiveness scores were calculated based on each participant's inverse reaction time (IRT [1/RT]) as described below. Sleep intervals derived from participants' actigraphy data—or from sleep diaries, if available, where actigraphy data were missing—were used to calculate modeled healthy control task effectiveness scores using the SAFTE. [38,39] Participants evidenced good sleep hygiene, with minimal missing data. Task effectiveness was not modeled for participants missing data from the night before PVT testing. A SAFTE model effectiveness score of 100 indicates typical best performance during the day, based on a normal healthy population. SAFTE-FAST effectiveness predictions have been validated against IRT on the PVT [22].

### Statistical Analyses

Target enrollment in the studies used to generate PVT data was based on the primary endpoint [33,34]. SAFTE modeling was an experimental endpoint and as such was not considered for sample size calculations.

SAFTE-FAST modeled task effectiveness based on measured sleep served as a proxy for performance measures from healthy controls, referred to here as “modeled healthy control task effectiveness.” Sleep intervals exported from the Actiware software-date and time of each recorded sleep interval's start and end-were used in SAFTE-FAST to determine a continuous estimate of modeled healthy control task effectiveness across the entire study period. As SAFTE-FAST generates a continuous estimate of task effectiveness, modeled healthy control task effectiveness scores from the time when PVTs were taken were identified using time stamp data from the PVT. Synchronized task effectiveness scores were exported from SAFTE-FAST for subsequent comparison against actual effectiveness.

Actual effectiveness scores were calculated as a percentage of a theoretical “best performance” from participants' average IRT per PVT trial (for all PVT test sessions, actual effectiveness=IRT/3.96×100). Average speed (IRT) on a PVT under optimal conditions—healthy sleepers taking the test at 11:00 in the morning, following an 8-hour sleep opportunity—is approximately 3.96 [40], and is assumed to represent an actual effectiveness score of 100 (a healthy participant's typical best performance). Repeated-measures analysis of variance (ANOVA) was used to estimate the effect of treatment on actual effectiveness over time.

For additional analyses of data from model input parameters, repeated-measures ANOVA was performed to compare the effects of treatment on PVT measures (IRT, MRT, lapses, and errors). Fixed effects included treatment, time, and treatment×time interaction. Treatment order; habitual TIB, TST, and DSIs; and previous day's TIB, TST, and DSIs were treated as covariates, while participant was modeled as a random effect. Post hoc tests (marginal means, pairwise comparisons) were performed to compare PVT measures between each trial. Mean habitual sleep parameters and sleep parameters obtained the day before PVT testing were compared across all conditions and with those obtained during the baseline period using Student's t tests, assuming unequal variance. The anticipated risk reduction associated with solriamfetol was computed as the difference between PVT performance metrics by treatment condition. All statistical analyses were performed with Stata 15.1 and Excel 2016. P values were not controlled for multiplicity and therefore are nominal.

TABLE 11
Actual^{a} and modeled healthy control task effectiveness scores by treatment

| | | | | | ANOVA P Values^{b} | | |
|---|---|---|---|---|---|---|---|
| | | | LS Mean | | | | Previous |
| | LS Mean (SE) | | Difference | P | | Treatment × | Day's |
| | Placebo | Solriamfetol | (95% CI) | Value^{b} | Treatment | Time | Sleep |
| OSA | | | | | | | |
| Actual task | (n = 31) | (n = 31) | | | 0.68 | 0.24 | 0.001 |
| effectiveness | | | | | | | |
| Pre-dose | 93.64 (1.17) | 89.19 (1.12) | −4.45 (−8.15, | 0.02 | | | |
| | | | −0.75) | | | | |
| 2 hours post-dose | 94.23 (1.17) | 91.88 (1.12) | −2.35 (−6.06, | 0.21 | | | |
| | | | 1.34) | | | | |
| 6 hours post-dose | 95.07 (1.17) | 93.72 (1.12) | −1.35 (−5.05, | 0.47 | | | |
| | | | 2.36) | | | | |
| Modeled healthy | (n = 31) | (n = 31) | | | 0.10 | 0.92 | 0.04 |
| control task | | | | | | | |
| effectiveness | | | | | | | |
| Pre-dose | 95.28 (0.19) | 93.27 (0.18) | −2.01 (−2.62, | <0.001 | | | |
| | | | −1.41) | | | | |
| 2 hours post-dose | 96.13 (0.19) | 94.11 (0.18) | −2.02 (−2.61, | <0.001 | | | |
| | | | −1.41) | | | | |
| 6 hours post-dose | 93.46 (0.19) | 91.56 (0.18) | −1.90 (−2.51, | <0.001 | | | |
| | | | −1.31) | | | | |
| Narcolepsy | | | | | | | |
| Actual task | (n = 20) | (n = 20) | | | 0.31 | <0.001 | 0.004 |
| effectiveness | | | | | | | |
| Pre-dose | 92.23 (1.46) | 86.06 (1.65) | −6.16 (−10.59, | 0.007 | | | |
| | | | −1.73) | | | | |
| 2 hours post-dose | 85.66 (1.46) | 96.07 (1.65) | 10.41 (5.97, | <0.001 | | | |
| | | | 14.83) | | | | |
| 6 hours post-dose | 86.29 (1.46) | 93.89 (1.65) | 7.60 (3.17, | 0.001 | | | |
| | | | 12.03) | | | | |
| Modeled healthy | (n = 20) | (n = 20) | | | 0.19 | 0.88 | <0.001 |
| control task | | | | | | | |
| effectiveness | | | | | | | |
| Pre-dose | 94.39 (0.19) | 93.49 (0.22) | −0.90 (−1.48, | 0.003 | | | |
| | | | −0.32) | | | | |
| 2 hours post-dose | 95.71 (0.19) | 94.63 (0.22) | −1.08 (−1.65, | <0.001 | | | |
| | | | −0.49) | | | | |
| 6 hours post-dose | 93.41 (0.19) | 92.35 (0.22) | −1.06 (−1.64, | <0.001 | | | |
| | | | −0.48) | | | | |

ANOVA, analysis of variance; CI, confidence interval; IRT, inverse reaction time; LS, least squares; OSA, obstructive sleep apnea; SE, standard error.
^{a}Actual effectiveness calculated as IRT/3.96 × 100.
^{b}P values are nominal.

TABLE 12
PVT results in participants with OSA

| | | | | | ANOVA P Values^{a} | | |
|---|---|---|---|---|---|---|---|
| | LS Mean (SE) | | LS Mean | | | | Previous |
| | Placebo | Solriamfetol | Difference | P | | Treatment × | Day's |
| | (n = 31) | (n = 31) | (95% CI) | Value^{a} | Treatment | Time | Sleep |
| Inverse | | | | | 0.68 | 0.24 | 0.001 |
| reaction time, | | | | | | | |
| 1/ms | | | | | | | |
| Pre-dose | 3.71 (0.05) | 3.53 (0.04) | −0.18 (−0.32, | 0.02 | | | |
| | | | −0.03) | | | | |
| 2 hours post- | 3.73 (0.05) | 3.64 (0.04) | −0.09 (−0.24, | 0.21 | | | |
| dose | | | 0.05) | | | | |
| 6 hours post- | 3.76 (0.05) | 3.71 (0.04) | −0.05 (−0.20, | 0.47 | | | |
| dose | | | 0.09) | | | | |
| Mean reaction | | | | | 0.31 | 0.25 | 0.02 |
| time, ms | | | | | | | |
| Pre-dose | 296 (6.22) | 303 (5.94) | 6.5 (−13.2, | 0.52 | | | |
| | | | 26.2) | | | | |
| 2 hours post- | 296 (6.22) | 292 (5.94) | −3.96 (−23.7, | 0.69 | | | |
| dose | | | 15.7) | | | | |
| 6 hours post- | 302 (6.22) | 292 (5.94) | −9.84 (−29.5, | 0.32 | | | |
| dose | | | 9.9) | | | | |
| Lapses, n/trial | | | | | 0.34 | 0.91 | 0.49 |
| Pre-dose | 5.95 (0.94) | 3.82 (0.90) | −2.13 (−5.11, | 0.16 | | | |
| | | | 0.85) | | | | |
| 2 hours post- | 5.66 (0.94) | 3.02 (0.90) | −2.64 (−5.62, | 0.08 | | | |
| dose | | | 0.34) | | | | |
| 6 hours post- | 6.04 (0.94) | 3.98 (0.90) | −2.06 (−5.04, | 0.17 | | | |
| dose | | | 0.92) | | | | |
| Errors, n/trial | | | | | 0.78 | 0.77 | 1.00 |
| Pre-dose | 1.10 (0.61) | 1.24 (0.58) | 0.14 (−1.79, | 0.89 | | | |
| | | | 2.07) | | | | |
| 2 hours post- | 2.39 (0.61) | 1.84 (0.58) | −0.55 (−2.48, | 0.57 | | | |
| dose | | | 1.38) | | | | |
| 6 hours post- | 1.91 (0.61) | 1.84 (0.58) | −0.07 (−2.00, | 0.95 | | | |
| dose | | | 1.86) | | | | |

ANOVA, analysis of variance; CI, confidence interval; LS, least squares; OSA, obstructive sleep apnea; PVT, psychomotor vigilance task; SE, standard error.
^{a}P values are nominal.

TABLE 13
PVT results in participants with narcolepsy

| | | | | | ANOVA P Value^{a} | | |
|---|---|---|---|---|---|---|---|
| | LS Mean (SE) | | LS Mean | | | | Previous |
| | Placebo | Solriamfetol | Difference | P | | Treatment × | Day's |
| | (n = 20) | (n = 20) | (95% CI) | Value^{a} | Treatment | Time | Sleep |
| Inverse | | | | | 0.31 | <0.001 | 0.004 |
| reaction time, | | | | | | | |
| 1/ms | | | | | | | |
| Pre-dose | 3.65 (0.06) | 3.41 (0.07) | −0.24 (−0.42, | 0.007 | | | |
| | | | −0.07) | | | | |
| 2 hours post- | 3.39 (0.06) | 3.80 (0.07) | 0.41 (0.24, | <0.001 | | | |
| dose | | | 0.59) | | | | |
| 6 hours post- | 3.42 (0.06) | 3.72 (0.07) | 0.30 (0.13, | 0.001 | | | |
| dose | | | 0.48) | | | | |
| Mean | | | | | 0.52 | 0.06 | 0.91 |
| reaction time, | | | | | | | |
| ms | | | | | | | |
| Pre-dose | 388 (131.2) | 582 (148.4) | 194 (−204.2, | 0.34 | | | |
| | | | 591.4) | | | | |
| 2 hours post- | 699 (131.2) | 317 (148.4) | −382 (−779.9, | 0.06 | | | |
| dose | | | 15.7) | | | | |
| 6 hours post- | 657 (131.2) | 319 (148.4) | −338 (−735.8, | 0.10 | | | |
| dose | | | 59.8) | | | | |
| Lapses, | | | | | 0.59 | 0.03 | 0.65 |
| n/trial | | | | | | | |
| Pre-dose | 4.36 (1.42) | 6.63 (1.61) | 2.27 (−2.04, | 0.30 | | | |
| | | | 6.58) | | | | |
| 2 hours post- | 7.91 (1.42) | 3.35 (1.61) | −4.56 (−8.87, | 0.04 | | | |
| dose | | | −0.25) | | | | |
| 6 hours post- | 8.96 (1.42) | 4.52 (1.61) | −4.44 (−8.75, | 0.04 | | | |
| dose | | | −0.13) | | | | |
| Errors, n/trial | | | | | 0.50 | 0.92 | 1.00 |
| Pre-dose | 1.53 (0.37) | 1.11 (0.42) | −0.42 (−1.55, | 0.47 | | | |
| | | | 0.73) | | | | |
| 2 hours | 1.73 (0.37) | 1.39 (0.42) | −0.34 (−1.47, | 0.57 | | | |
| post-dose | | | 0.80) | | | | |
| 6 hours | 1.98 (0.37) | 1.34 (0.32) | −0.64 (−1.78, | 0.26 | | | |
| post-dose | | | 0.49) | | | | |

ANOVA, analysis of variance; CI, confidence interval; LS, least squares; PVT, psychomotor vigilance task; SE, standard error.
^{a}P values are nominal

### REFERENCES

[1] Gandhi K D, Mansukhani M P, Silber M H, Kolla B P. Excessive daytime sleepiness: a clinical review. Mayo Clin Proc. 2021; 96(5):1288-301.
[2] Seneviratne U, Puvanendran K. Excessive daytime sleepiness in obstructive sleep apnea: prevalence, severity, and predictors. Sleep Med. 2004; 5(4):339-43.
[3] Bjorvatn B, Lehmann S, Gulati S, Aurlien H, Pallesen S, Saxvig I W. Prevalence of excessive sleepiness is higher whereas insomnia is lower with greater severity of obstructive sleep apnea. Sleep Breath. 2015; 19(4):1387-93.
[4] Gasa M, Tamisier R, Launois S H, Sapene M, Martin F, Stach B, et al. Residual sleepiness in sleep apnea patients treated by continuous positive airway pressure. J Sleep Res. 2013; 22(4):389-97.
[5] Pepin J L, Viot-Blanc V, Escourrou P, Racineux J L, Sapene M, Levy P, et al. Prevalence of residual excessive sleepiness in CPAP-treated sleep apnoea patients: the French multicentre study. Eur Respir J. 2009; 33(5):1062-7.
[6] Van Schie M K, Thijs R D, Fronczek R, Middelkoop H A, Lammers G J, Van Dijk J G. Sustained attention to response task (SART) shows impaired vigilance in a spectrum of disorders of excessive daytime sleepiness. J Sleep Res. 2012; 21(4):390-5.
[7] Gharibi V, Mokarami H, Cousins R, Jahangiri M, Eskandari D. Excessive daytime sleepiness and safety performance: comparing proactive and reactive approaches. Int J Occup Environ Med. 2020; 11(2):95-107.
[8] Ward K L, Hillman D R, James A, Bremner A P, Simpson L, Cooper M N, et al. Excessive daytime sleepiness increases the risk of motor vehicle crash in obstructive sleep apnea. J Clin Sleep Med. 2013; 9(10):1013-21.
[9] Lloberes P, Levy G, Descals C, Sampol G, Roca A, Sagales T, et al. Self-reported sleepiness while driving as a risk factor for traffic accidents in patients with obstructive sleep apnoea syndrome and in non-apnoeic snorers. Respir Med. 2000; 94(10):971-6.
[10] Philip P, Sagaspe P, Lagarde E, Leger D, Ohayon M M, Bioulac B, et al. Sleep disorders and accidental risk in a large group of regular registered highway drivers. Sleep Med. 2010; 11(10):973-9.
[11] Philip P, Bailly S, Benmerad M, Micoulaud-Franchi J A, Grillet Y, Sapene M, et al. Self-reported sleepiness and not the apnoea hypopnoea index is the best predictor of sleepiness-related accidents in obstructive sleep apnoea. Sci Rep. 2020; 10(1):16267.
[12] Philip P, Chaufton C, Taillard J, Capelli A, Coste O, Leger D, et al. Modafinil improves real driving performance in patients with hypersomnia: a randomized double-blind placebo-controlled crossover clinical trial. Sleep. 2014; 37(3):483-7.
[13] Kay G G, Feldman N. Effects of armodafinil on simulated driving and self-report measures in obstructive sleep apnea patients prior to treatment with continuous positive airway pressure. J Clin Sleep Med. 2013; 9(5):445-54.
[14] Li Y, Vgontzas A, Kritikou I, Fernandez-Mendoza J, Basta M, Pejovic S, et al. Psychomotor vigilance test and its association with daytime sleepiness and inflammation in sleep apnea: clinical implications. J Clin Sleep Med. 2017; 13(9):1049-56.
[15] Baulk S D, Biggs S N, Reid K J, van den Heuvel C J, Dawson D. Chasing the silver bullet: measuring driver fatigue using simple and complex tasks. Accid Anal Prev. 2008; 40(1):396-402.
[16] Thomann J, Baumann C R, Landolt H P, Werth E. Psychomotor vigilance task demonstrates impaired vigilance in disorders with excessive daytime sleepiness. J Clin Sleep Med. 2014; 10(9):1019-24.
[17] Jung C M, Ronda J M, Czeisler C A, Wright K P, Jr. Comparison of sustained attention assessed by auditory and visual psychomotor vigilance tasks prior to and during sleep deprivation. J Sleep Res. 2011; 20(2):348-55.
[18] Chua E C, Sullivan J P, Duffy J F, Klerman E B, Lockley S W, Kristal B S, et al. Classifying attentional vulnerability to total sleep deprivation using baseline features of psychomotor vigilance test performance. Sci Rep. 2019; 9(1):12102.
[19] Basner M, Dinges D F. Maximizing sensitivity of the psychomotor vigilance test (PVT) to sleep loss. Sleep. 2011; 34(5):581-91.
[20] Santhi N, Horowitz T S, Duffy J F, Czeisler C A. Acute sleep deprivation and circadian misalignment associated with transition onto the first night of work impairs visual selective attention. PLoS One. 2007; 2(11):e1233.
[21] Hursh S R, Redmond D P, Johnson M L, Thorne D R, Belenky G, Balkin T J, et al. Fatigue models for applied research in warfighting. Aviat Space Environ Med. 2004; 75(3 suppl):A44-A53.
[22] Hursh S, Balkin T, Miller J, Eddy D. The Fatigue Avoidance Scheduling Tool: modeling to minimize the effects of fatigue on cognitive performance. SAE Tech Paper. 2004; 2004-01-2151.
[23] Hursh S, Fanzone J F, Raslear T G. Analysis of the relationship between operator effectiveness measures and economic impacts of rail accidents Washington, DC: Federal Railroad Administration; 2011 [May 2011]. Available from: https://railroads.dot.gov/elibrary/analysis-relationship-between-operator-effectiveness-measures-and-economic-impacts-rail.
[24] Sunosi™ (solriamfetol) tablets Prescribing Information. Palo Alto, CA: Jazz Pharmaceuticals, Inc; 221 October 2021.
[25] Sunosi™ (solriamfetol) tablets Summary of Product Characteristics. Dublin, Ireland: Jazz Pharmaceuticals Ireland Ltd; 220 January 2020.
[26] Schweitzer P K, Rosenberg R, Zammit G K, Gotfried M, Chen D, Carter L P, et al. Solriamfetol for excessive sleepiness in obstructive sleep apnea (TONES 3): a randomized controlled trial. Am J Respir Crit Care Med. 2019; 199(11):1421-31.
[27] Strollo P J, Jr., Hedner J, Collop N, Lorch D G, Jr., Chen D, Carter L P, et al. Solriamfetol for the treatment of excessive sleepiness in OSA: a placebo-controlled randomized withdrawal study. Chest. 2019; 155(2):364-74.
[28] Thorpy M J, Shapiro C, Mayer G, Corser B C, Emsellem H, Plazzi G, et al. A randomized study of solriamfetol for excessive sleepiness in narcolepsy. Ann Neurol. 2019; 85(3):359-70.
[29] Emsellem H A, Thorpy M J, Lammers G J, Shapiro C M, Mayer G, Plazzi G, et al. Measures of functional outcomes, work productivity, and quality of life from a randomized, phase 3 study of solriamfetol in participants with narcolepsy. Sleep Med. 2020; 67:128-36.
[30] Malhotra A, Shapiro C, Pepin J L, Hedner J, Ahmed M, Foldvary-Schaefer N, et al. Long-term study of the safety and maintenance of efficacy of solriamfetol (JZP-110) in the treatment of excessive sleepiness in participants with narcolepsy or obstructive sleep apnea. Sleep. 2020; 43(2):zsz220.
[31] Weaver T E, Drake C L, Benes H, Stern T, Maynard J, Thein S G, et al. Effects of solriamfetol on quality of life measures from a 12-week phase 3 randomized trial. Ann Am Thorac Soc. 2020; 17(8):998-1007.
[32] Weaver T E, Pepin J L, Schwab R, Shapiro C, Hedner J, Ahmed M, et al. Long-term effects of solriamfetol on quality of life and work productivity in participants with excessive daytime sleepiness associated with narcolepsy or obstructive sleep apnea. J Clin Sleep Med. 2021; 17(10):1995-2007.
[33] Vinckenbosch F, Lammers G, Overeem S, Chen D, Wang G, Carter L, et al. Effects of solriamfetol on driving performance in participants with narcolepsy [abstract 0763]. Sleep. 2020; 43(suppl):A290.
[34] Vinckenbosch F, Asin J, de Vries N, Vonk P E, Donjacour C, Lammers G J, et al. Effects of solriamfetol on on-the-road driving performance in participants with excessive daytime sleepiness associated with obstructive sleep apnoea. Hum Psychopharmacol. 2022:e2845.
[35] Bogan R K, Feldman N, Emsellem H A, Rosenberg R, Lu Y, Bream G, et al. Effect of oral JZP-110 (ADX-N05) treatment on wakefulness and sleepiness in adults with narcolepsy. Sleep Med. 2015; 16(9):1102-8.
[36] Ruoff C, Swick T J, Doekel R, Emsellem H A, Feldman N T, Rosenberg R, et al. Effect of oral JZP-110 (ADX-N05) on wakefulness and sleepiness in adults with narcolepsy: a phase 2b study. Sleep. 2016; 39(7):1379-87.
[37] Ancoli-Israel S, Martin J L, Blackwell T, Buenaver L, Liu L, Meltzer L J, et al. The SBSM guide to actigraphy monitoring: clinical and research applications. Behav Sleep Med. 2015; 13(suppl 1):S4-S38.
[38] Rawlings Hursh S, Elsmore T, Eddy D, Inventors. Interface for a system and method for evaluating task effectiveness based on sleep patterns. U.S. Pat. No. 7,118,530. Oct. 10, 2006: Science Applications International Corp., assignee.; 2006 [Available from: https://patents.google.com/patent/U.S. Pat. No. 7,118,530B2/en.
[39] Rawlings Hursh S, Inventor. Evaluating task effectiveness based on sleep pattern. U.S. Pat. No. 7,207,938. Apr. 24, 2007: Science Applications International Corporation, assignee.; 2007 [Available from: https://patents.google.com/patent/U.S. Pat. No. 7,207,938B2/en.
[40] Jongen S, Perrier J, Vuurman E F, Ramaekers J G, Vermeeren A. Sensitivity and validity of psychometric tests for assessing driving impairment: effects of sleep deprivation. PLoS One. 2015; 10(2):e0117045.
[41] Batool-Anwar S, Kales S N, Patel S R, Varvarigou V, DeYoung P N, Malhotra A. Obstructive sleep apnea and psychomotor vigilance task performance. Nat Sci Sleep. 2014; 6:65-71.
[42] Owens K, Ramaekers J G. Drugs, driving, and models to measure driving impairment. In: Verster J C, Pandi-Perumal S R, Ramaekers J G, de Gier J J, editors. Drugs, Driving and Traffic Safety. Basel, Switzerland: Birkhauser Verlag; 2009. p. 43-58.
[43] Broughton W A, Broughton R J. Psychosocial impact of narcolepsy. Sleep. 1994; 17(8 suppl):S45-S9.
[44] Flightcrew member duty and rest requirements: final rule. Fed Regist. 2012; 77(2):329-403.
[45] Liu S Y, Perez M A, Lau N. The impact of sleep disorders on driving safety—findings from the Second Strategic Highway Research Program naturalistic driving study. Sleep. 2018; 41(4):1-11.
[46] Pizza F, Jaussent I, Lopez R, Pesenti C, Plazzi G, Drouot X, et al. Car crashes and central disorders of hypersomnolence: a French study. PLoS One. 2015; 10(6):e0129386.
[47] Tzeng N S, Hsing S C, Chung C H, Chang H A, Kao Y C, Mao W C, et al. The risk of hospitalization for motor vehicle accident injury in narcolepsy and the benefits of stimulant use: a nationwide cohort study in Taiwan. J Clin Sleep Med. 2019; 15(6):881-9.
[48] Tregear S, Reston J, Schoelles K, Phillips B. Obstructive sleep apnea and risk of motor vehicle crash: systematic review and meta-analysis. J Clin Sleep Med. 2009; 5(6):573-81.
[49] Zomorodi K, Kankam M, Lu Y. A phase I, randomized, crossover, open-label study of the pharmacokinetics of solriamfetol (JZP-110) in healthy adult subjects with and without food. Clin Ther. 2019; 41(2):196-204.

The foregoing is illustrative of the present invention and is not to be construed as limiting thereof. The invention is defined by the following claims, with equivalents of the claims to be included therein. All publications, patent applications, patents, patent publications, and any other references cited herein are incorporated by reference in their entireties for the teachings relevant to the sentence and/or paragraph in which the reference is presented.

1.-12. (canceled)

13. A method of improving on-the-road driving performance in a subject in need thereof, said method comprising administering to the subject a pharmaceutically effective amount of [R]-2-amino-3-phenylpropylcarbamate (APC) or a pharmaceutically acceptable salt thereof, thereby improving on-the-road driving performance in the subject.

14. The method of claim 13, wherein the subject is provided with APC or a pharmaceutically acceptable salt thereof in a daily dose equivalent to about 37.5 mg to about 300 mg APC.

15. The method of claim 13, wherein the APC or a pharmaceutically acceptable salt thereof is APC hydrochloride.

16. The method of claim 13, wherein the APC or a pharmaceutically acceptable salt thereof is administered in a dose escalation regimen over at least 7 days.

17. The method of claim 13, wherein the subject has excessive daytime sleepiness.

18. The method of claim 17, wherein the excessive daytime sleepiness is associated with narcolepsy or obstructive sleep apnea.

19. The method of claim 17, wherein the excessive daytime sleepiness is associated with depression.

20. The method of claim 13, wherein the on-the-road driving performance is assessed by measuring the standard deviation of lateral position (SDLP), standard deviation of speed, and/or number of lane drifts while operating a vehicle.

21. The method of claim 20, wherein the on-the-road driving performance is measured over the course of about 30 minutes to about 120 minutes.

22. The method of claim 20, wherein the on-the-road driving performance is assessed from about 1 hour after providing APC to the subject, to about 12 hours after administering APC to the subject.

23. The method of claim 20, wherein the on-the-road driving performance as assessed by measuring the SDLP improves from about 1.0 cm to about 15 cm after providing APC to the subject.

24. The method of claim 13, wherein the subject is a human.
